(19) United States
(12) Patent Application Publication
(10) Pub. No.: US 2023/0146653 A1
(43) Pub. Date: May 11, 2023

(54) HETEROCYCLIC COMPOUND AND ORGANIC LIGHT-EMITTING ELEMENT COMPRISING SAME

(71) Applicant: LG CHEM, LTD., Seoul (KR)

(72) Inventors: Hanbit JANG, Daejeon (KR); Seongmi CHO, Daejeon (KR); Hoyong LEE, Daejeon (KR); Seonkyoung SON, Daejeon (KR); Jaemyeng JEONG, Daejeon (KR); Seoyoung IM, Daejeon (KR)

(21) Appl. No.: 17/912,154

(22) PCT Filed: Apr. 15, 2021
(86) PCT No.: PCT/KR2021/004760

§ 371 Date: Sep. 16, 2022

(30) Foreign Application Priority Data
Apr. 16, 2020 (KR) 10-2020-0045970

(51) Int. Cl. H10K 85/60; C07D 491/16; C09K 11/06; C07D 495/16

(52) U.S. Cl. CPC H10K 85/654; C07D 491/16; C09K 11/06; H10K 85/657; C07D 495/16; C09K 2211/1018; H10K 50/11

(57) ABSTRACT

Provided is a low sublimation temperature heterocyclic compound of Chemical Formula 1:

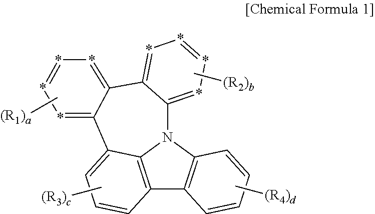

wherein, in Chemical Formula 1, adjacent one or more pairs of * bond to X₁ and X₂ of the following Chemical Formula 2:

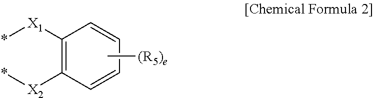

where * means the part of Chemical Formula 1 bonding to X₁ and X₂, and the substituents are as defined in the specification, and an organic light emitting device including the same, whereby the heterocyclic compound of Chemical Formula 1 increases efficiency and stability of the organic light emitting device.

### CROSS-REFERENCE TO RELATED APPLICATION(S)

This application is a National Stage Application of International Application No. PCT/KR2021/004760 filed on Apr. 15, 2021, which claims priority to and the benefits of Korean Patent Application No. 10-2020-0045970, filed with the Korean Intellectual Property Office on Apr. 16, 2020, the entire contents of which are incorporated herein by reference.

### TECHNICAL FIELD

The present specification relates to a heterocyclic compound, and an organic light emitting device including the same.

### BACKGROUND

In the present specification, an organic light emitting device is a light emitting device using an organic semiconductor material, and requires an exchange of holes and/or electrons between an electrode and the organic semiconductor material. An organic light emitting device can be largely divided into two types as follows depending on the operation principle. The first is a light emitting device type in which excitons are formed in an organic material layer by photons introduced to a device from an external light source, these excitons are separated into electrons and holes, and these electrons and holes are each transferred to different electrodes and used as a current source (voltage source). The second is a light emitting device type in which, by applying a voltage or current to two or more electrodes, holes and/or electrons are injected into an organic semiconductor material layer forming an interface with the electrodes, and the light emitting device is operated by the injected electrons and holes.

An organic light emission phenomenon generally refers to a phenomenon converting electrical energy to light energy using an organic material. An organic light emitting device using an organic light emission phenomenon normally has a structure including an anode, a cathode, and an organic material layer therebetween. Herein, the organic material layer is often formed in a multilayer structure formed with different materials in order to increase efficiency and stability of the organic light emitting device, and for example, can be formed with a hole injection layer, a hole transfer layer, a light emitting layer, an electron blocking layer, an electron transfer layer, an electron injection layer and the like. When a voltage is applied between the two electrodes in such an organic light emitting device structure, holes and electrons are injected to the organic material layer from the anode and the cathode, respectively, and when the injected holes and electrons meet, excitons are formed, and light emits when these excitons fall back to the ground state. Such an organic light emitting device is known to have properties such as self-emission, high luminance, high efficiency, low driving voltage, wide viewing angle and high contrast.

Materials used as an organic material layer in an organic light emitting device can be divided into a light emitting material and a charge transfer material, for example, a hole injection material, a hole transfer material, an electron blocking material, an electron transfer material, an electron injection material and the like depending on the function. The light emitting material includes, depending on light emitting color, blue, green and red light emitting materials, and yellow and orange light emitting materials required for obtaining better natural colors.

In addition, in order to increase color purity and increase light emission efficiency through energy transfer, a host/dopant system can be used as a light emitting material. The principal is that, when a small amount of a dopant having a smaller energy band gap and having excellent light emission efficiency compared to a host that forms most of a light emitting layer is mixed to the light emitting layer, excitons generated in the host are transferred to the dopant, and light with high efficiency emits. Herein, a wavelength of the host moves to a wavelength band of the dopant, and therefore, light with a target wavelength can be obtained depending on the types of the dopant used.

In order to fully exhibit excellent properties that the organic light emitting device described above has, materials forming an organic material layer in the device, for example, a hole injection material, a hole transfer material, a light emitting material, an electron blocking material, an electron transfer material, an electron injection material and the like, are supported by stable and efficient materials, and accordingly, development of new materials has been continuously required.

### BRIEF DESCRIPTION

### Technical Problem

The present specification describes a heterocyclic compound, and an organic light emitting device including the same.

### Technical Solution

One embodiment of the present disclosure provides a heterocyclic compound of the following Chemical Formula 1:

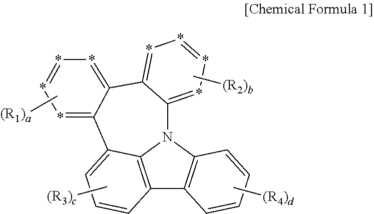

wherein in Chemical Formula 1:

adjacent one or more pairs of * bond to X₁ and X₂ of the following Chemical Formula 2:

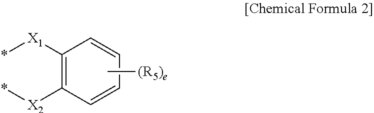

wherein in Chemical Formula 2:

* means the part of Chemical Formula 1 bonding to X₁ and X₂;

any one of X₁ and X₂ is a direct bond;

when X₁ is a direct bond, X₂ is CRaRb, O or S;

when X₂ is a direct bond, X₁ is CRaRb, O or S, and at least one of R₁ to R₅ is the following Chemical Formula 3, and the rest that are not Chemical Formula 3 are the same as or different from each other and each independently is hydrogen, deuterium, a substituted or unsubstituted alkyl group, a substituted or unsubstituted aryl group, a substituted or unsubstituted heteroaryl group, a substituted or unsubstituted cycloalkyl group, a substituted or unsubstituted alkoxy group or a combination thereof;

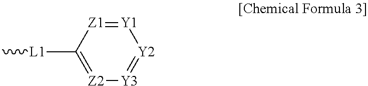

wherein in Chemical Formula 3:

[symbol] is a part bonding to the core of Chemical Formula 1;

at least one of Z1 and Z2 is N, and the other one is CRc;

Y1 to Y3 are the same as or different from each other, and each independently is CRd or N;

L1 is a direct bond, a substituted or unsubstituted alkylene group, a substituted or unsubstituted arylene group, a substituted or unsubstituted heteroarylene group or a combination thereof;

Ra to Rd are the same as or different from each other, and each independently is hydrogen, deuterium, a substituted or unsubstituted alkyl group, a substituted or unsubstituted aryl group, a substituted or unsubstituted heteroaryl group, a substituted or unsubstituted cycloalkyl group, a substituted or unsubstituted alkoxy group or a combination thereof;

Rd can bond to adjacent substituents to form a substituted or unsubstituted ring;

a is an integer of 0 to 4, and when a is 2 or greater, R₁s are the same as or different from each other;

b is an integer of 0 to 4, and when b is 2 or greater, R₂s are the same as or different from each other;

c is an integer of 0 to 3, and when c is 2 or greater, R₃s are the same as or different from each other;

d is an integer of 0 to 4, and when d is 2 or greater, R₄s are the same as or different from each other; and

e is an integer of 0 to 4, and when e is 2 or greater, R₅s are the same as or different from each other.

In addition, one embodiment of the present disclosure provides an organic light emitting device including a first electrode; a second electrode provided opposite to the first electrode; and one or more organic material layers provided between the first electrode and the second electrode, wherein one or more layers of the organic material layers include the heterocyclic compound described above.

### Advantageous Effects

A heterocyclic compound of the present disclosure can be used as a material of an organic material layer of an organic light emitting device. When manufacturing an organic light emitting device including the heterocyclic compound of the present disclosure, an organic light emitting device having high efficiency, low voltage and long lifetime properties can be obtained, and when including the heterocyclic compound of the present disclosure in a light emitting layer of an organic light emitting device, an organic light emitting device having high color gamut can be manufactured.

### DESCRIPTION OF THE DRAWINGS

FIG. 1 to FIG. 3 each illustrate an example of an organic light emitting device according to the present disclosure.

### REFERENCE NUMERALS

1: Substrate
2: First Electrode
3: Light Emitting Layer
4: Second Electrode
5: Hole Injection Layer
6: Hole Transfer Layer
7: Electron Blocking Layer
8: Electron Transfer Layer
9: Electron Injection Layer
10: Electron Injection and Transfer Layer

### DETAILED DESCRIPTION

Hereinafter, the present specification will be described in more detail.

The present specification provides a heterocyclic compound of Chemical Formula 1. When using the heterocyclic compound of Chemical Formula 1 in an organic material layer of an organic light emitting device, efficiency and lifetime properties are enhanced in the organic light emitting device. Particularly, existing compounds having a high sublimation temperature have low compound stability causing problems of reducing device efficiency and lifetime when used in a device, however, by including a cycloalkene ring in the molecule, the heterocyclic compound of Chemical Formula 1 has a low sublimation temperature and thereby has high stability, and accordingly, a device having superior efficiency and long lifetime properties can be obtained when used in a device.

In addition, the heterocyclic compound of Chemical Formula 1 has increased solubility by including a cycloalkene ring in the molecule, and can also be used for a solution process.

In the present specification, a description of a certain part “including” certain constituents means capable of further including other constituents, and does not exclude other constituents unless particularly stated on the contrary.

In the present specification, a description of a certain member being placed “on” another member includes not only a case of the certain member being in contact with the another member but a case of still another member being present between the two members.

Examples of substituents in the present specification are described below, however, the substituents are not limited thereto.

The term “substitution” means a hydrogen atom bonding to a carbon atom of a compound is changed to another substituent, and the position of substitution is not limited as long as it is a position at which the hydrogen atom is substituted, that is, a position at which a substituent is capable of substituting, and when two or more substituents substitute, the two or more substituents can be the same as or different from each other.

The term “substituted or unsubstituted” in the present specification means being substituted with one, two or more substituents selected from the group consisting of deuterium, a halogen group, a cyano group (—CN), a silyl group, a boron group, a substituted or unsubstituted alkyl group, a substituted or unsubstituted alkoxy group, a substituted or unsubstituted cycloalkyl group, a substituted or unsubstituted aryl group, and a substituted or unsubstituted heterocyclic group, or being substituted with a substituent linking two or more substituents among the substituents illustrated above, or having no substituents. For example, the “substituent linking two or more substituents” can include a biphenyl group. In other words, a biphenyl group can be an aryl group, or interpreted as a substituent linking two phenyl groups.

Examples of the substituents are described below, however, the substituents are not limited thereto.

In the present specification, examples of the halogen group can include fluorine (F), chlorine (Cl), bromine (Br) or iodine (I).

In the present specification, the silyl group can be a chemical formula of —SiY11Y12Y13, and Y11, Y12 and Y13 can each be hydrogen, a substituted or unsubstituted alkyl group, or a substituted or unsubstituted aryl group. Specific examples of the silyl group can include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but are not limited thereto.

In the present specification, the boron group can be a chemical formula of —BY4Y5, and Y4 and Y5 can each be hydrogen, a substituted or unsubstituted alkyl group, or a substituted or unsubstituted aryl group. Specific examples of the boron group can include a trimethylboron group, a triethylboron group, a t-butyldimethylboron group, a triphenylboron group, a phenylboron group, and the like, but are not limited thereto.

In the present specification, the alkyl group can be linear or branched, and although not particularly limited thereto, the number of carbon atoms is preferably from 1 to 60. According to one embodiment, the number of carbon atoms of the alkyl group is from 1 to 30. According to another embodiment, the number of carbon atoms of the alkyl group is from 1 and 20. According to another embodiment, the number of carbon atoms of the alkyl group is from 1 to 10. Specific examples of the alkyl group can include a methyl group, an ethyl group, a propyl group, an isopropyl group, a butyl group, an isobutyl group, a tert-butyl group, a pentyl group, a hexyl group, a heptyl group, an octyl group, and the like, but are not limited thereto.

In the present specification, the alkyl group in the alkylthioxy group, the alkylsulfoxy group and the N-alkylheteroarylamine group is the same as the examples of the alkyl group described above. Specific examples of the alkylthioxy group can include a methylthioxy group, an ethylthioxy group, a tert-butylthioxy group, a hexylthioxy group, an octylthioxy group, and the like, and specific examples of the alkylsulfoxy group can include mesyl, an ethylsulfoxy group, a propylsulfoxy group, a butylsulfoxy group, and the like, however, the alkylthioxy group and the alkylsulfoxy group are not limited thereto.

In the present specification, the cycloalkyl group is not particularly limited, but preferably has 3 to 60 carbon atoms, and according to one embodiment, the number of carbon atoms of the cycloalkyl group is from 3 to 30. According to another embodiment, the number of carbon atoms of the cycloalkyl group is from 3 to 20. According to another embodiment, the number of carbon atoms of the cycloalkyl group is from 3 to 6. Specific examples thereof can include a cyclopropyl group, a cyclobutyl group, a cyclopentyl group, a cyclohexyl group, a cycloheptyl group, a cyclooctyl group, and the like, but are not limited thereto.

In the present specification, the aryl group is not particularly limited, but preferably has 6 to 60 carbon atoms, and can be a monocyclic aryl group or a polycyclic aryl group. According to one embodiment, the number of carbon atoms of the aryl group is from 6 to 30. According to one embodiment, the number of carbon atoms of the aryl group is from 6 to 20. When the aryl group is a monocyclic aryl group, examples thereof can include a phenyl group, a biphenyl group, a terphenyl group, and the like, but are not limited thereto. Examples of the polycyclic aryl group can include a naphthyl group, an anthracenyl group, a phenanthrenyl group, a pyrenyl group, a perylenyl group, a triphenyl group, a chrysenyl group, a fluorenyl group, and the like, but are not limited thereto.

In the present specification, the heteroaryl group is a cyclic group including one or more of N, O, P, S, Si and Se as a heteroatom, and although not particularly limited thereto, the number of carbon atoms is preferably from 2 to 60. According to one embodiment, the number of carbon atoms of the heteroaryl group is from 2 to 30. Examples of the heteroaryl group can include a pyridine group, a pyrrole group, a pyrimidine group, a pyridazinyl group, a furan group, a thiophene group, an imidazole group, a pyrazole group, a dibenzofuran group, a dibenzothiophene group, a carbazole group, and the like, but are not limited thereto.

In the present specification, the alkylene group has the same definition as the alkyl group except for being a divalent group.

In the present specification, the arylene group has the same definition as the aryl group except for being a divalent group.

In the present specification, the heteroarylene group has the same definition as the heteroaryl group except for being a divalent group.

In the present specification, an “adjacent group” can mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting ortho positions in a benzene ring, and two substituents substituting the same carbon in an aliphatic ring can be interpreted as groups ‘adjacent’ to each other.

In the present specification, the meaning of, among substituents, “bonding to adjacent substituents to form a substituted or unsubstituted ring” can be bonding to adjacent groups to form a substituted or unsubstituted aliphatic ring, a substituted or unsubstituted aromatic ring, or a substituted or unsubstituted heteroring.

In the present specification, a “combination” means that two or more substituents that are the same as or different from each other are selected. For example, it means that, when a linking group is formed by a combination of substituents, the substituents that are the same as or different from each other are consecutively overlapped and linked (-phenylene group-phenylene group-, -naphthylene group-anthracenylene group-phenylene group-, and the like),

and, in the case of terminal substituents, means that a plurality of substituents are each substituted with substituents that are the same as or different from each other. For example, when one or more phenyl groups and one or more triazine groups each bond to one benzene ring, it can be interpreted that the corresponding benzene ring is substituted with a ‘combination’ of a phenyl group and a triazine group.

In the present specification, the descriptions of the aryl group can be used for the aromatic ring.

In the present specification, the descriptions of the heteroaryl group can be used for the heteroring.

In the present specification, the descriptions of the cycloalkyl group can be used for the aliphatic ring.

In the present specification, an alicyclic ring is a structure bonding in a ring shape, and means a ring that is not aromatic. Examples of the alicyclic ring can include cycloalkene, and the cycloalkene is, although a double bond is present in the hydrocarbon ring, a cyclic group that is not aromatic. Although not particularly limited thereto, the number of carbon atoms can be from 3 to 60, and according to one embodiment, the number of carbon atoms can be from 3 to 30. Examples of the cycloalkene can include cyclopropene, cyclobutene, cyclopentene, cyclohexene and the like, but are not limited thereto.

According to one embodiment of the present specification, Chemical Formula 1 is the following Chemical Formula 1-1 or Chemical Formula 1-2:

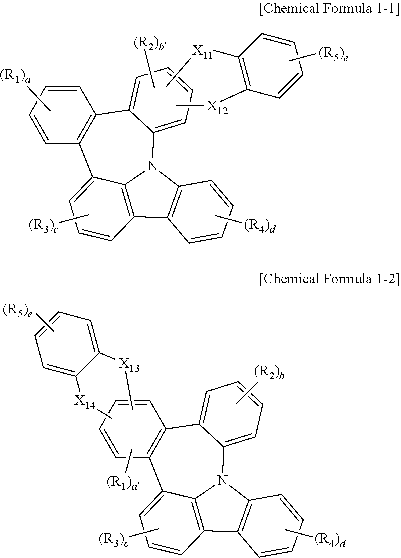

wherein in Chemical Formula 1-1 and Chemical Formula 1-2, a to e have the same definitions as in Chemical Formulae 1 and 2;

any one of X₁₁ and X₁₂ is a direct bond, and the other one is CRaRb, O or S;

any one of X₁₃ and X₁₄ is a direct bond, and the other one is CRaRb, O or S;

Ra and Rb are the same as or different from each other, and each independently is hydrogen, deuterium, a substituted or unsubstituted alkyl group, a substituted or unsubstituted aryl group, a substituted or unsubstituted heteroaryl group, a substituted or unsubstituted cycloalkyl group, a substituted or unsubstituted alkoxy group or a combination thereof; and

at least one of R₁ to R₅ is the following Chemical Formula 3, and the rest that are not Chemical Formula 3 are the same as or different from each other and each independently is hydrogen, deuterium, a substituted or unsubstituted alkyl group, a substituted or unsubstituted aryl group, a substituted or unsubstituted heteroaryl group, a substituted or unsubstituted cycloalkyl group, a substituted or unsubstituted alkoxy group or a combination thereof;

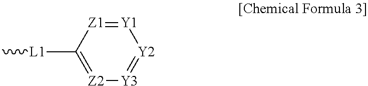

wherein in Chemical Formula 3:

[symbol] is a part bonding to the core of Chemical Formula 1-1 or 1-2;

at least one of Z1 and Z2 is N, and the other one is CRc;

Y1 to Y3 are the same as or different from each other, and each independently is CRd or N;

L1 is a direct bond, a substituted or unsubstituted alkylene group, a substituted or unsubstituted arylene group, a substituted or unsubstituted heteroarylene group, or a combination thereof;

Rc and Rd have the same definitions as above;

a′ is an integer of 0 to 2, and when a′ is 2 or greater, R₁s are the same as or different from each other; and

b′ is an integer of 0 to 2, and when b′ is 2 or greater, the R₂s are the same as or different from each other.

According to one embodiment of the present specification, Chemical Formula 1 is the following Chemical Formula 1-3.

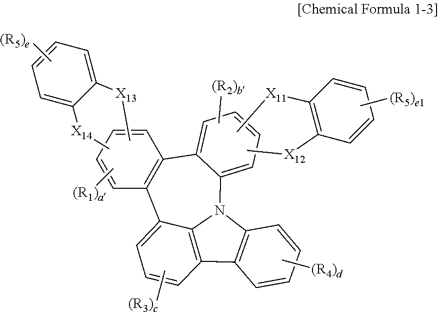

wherein in Chemical Formula 1-3, c to e have the same definitions as in Chemical Formulae 1 and 2;

any one of X₁₁ and X₁₂ is a direct bond, and the other one is CRaRb, O or S;

any one of X₁₃ and X₁₄ is a direct bond, and the other one is CRaRb, O or S;

Ra and Rb are the same as or different from each other, and each independently is hydrogen, deuterium, a substituted or unsubstituted alkyl group, a substituted or unsubstituted aryl group, a substituted or unsubstituted heteroaryl group, a substituted or unsubstituted cycloalkyl group, a substituted or unsubstituted alkoxy group or a combination thereof; and

at least one of R₁ to R₅ and R₁₅ is the following Chemical Formula 3, and the rest that are not Chemical Formula 3 are the same as or different from each other and each independently is hydrogen, deuterium, a substituted or unsubstituted alkyl group, a substituted or unsubstituted aryl group, a substituted or unsubstituted heteroaryl group, a substituted or unsubstituted cycloalkyl group, a substituted or unsubstituted alkoxy group or a combination thereof;

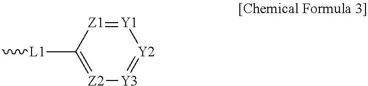

wherein in Chemical Formula 3:

[symbol] is a part bonding to the core of Chemical Formula 1-3;

at least one of Z1 and Z2 is N, and the other one is CRc;

Y1 to Y3 are the same as or different from each other, and each independently is CRd or N;

L1 is a direct bond, a substituted or unsubstituted alkylene group, a substituted or unsubstituted arylene group, a substituted or unsubstituted heteroarylene group, or a combination thereof;

Rc and Rd have the same definitions as above;

e1 is an integer of 0 to 4, and when e1 is 2 or greater, the R₁₅s are the same as or different from each other;

a′ is an integer of 0 to 2, and when a′ is 2 or greater, the R₁s are the same as or different from each other; and

b′ is an integer of 0 to 2, and when b′ is 2 or greater, the R₂s are the same as or different from each other.

According to one embodiment of the present specification, Chemical Formula 1 is any one of the following Chemical Formula 1-4 to Chemical Formula 1-11:

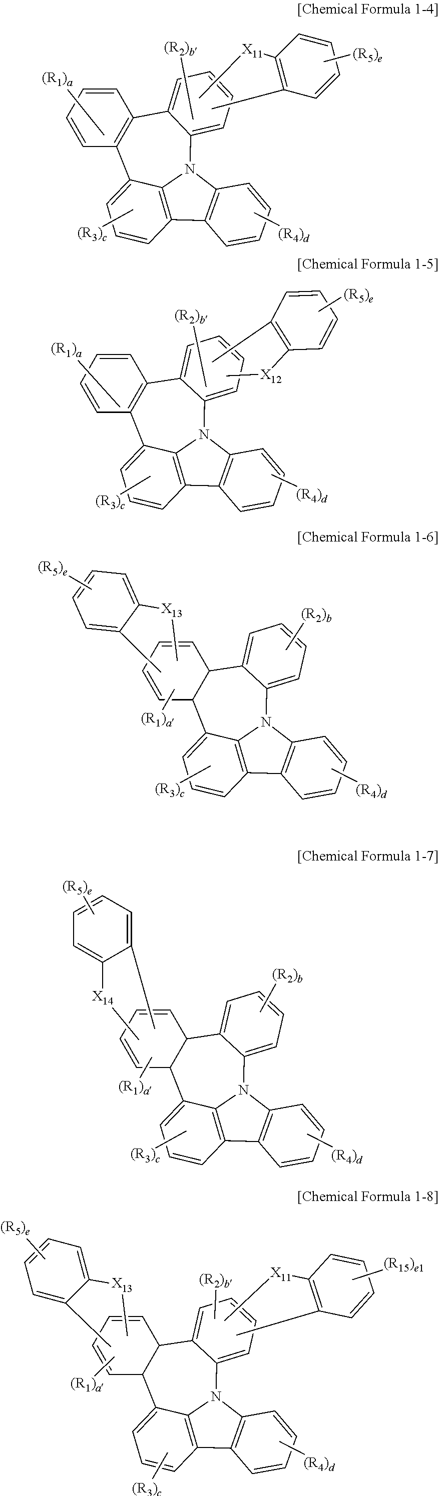

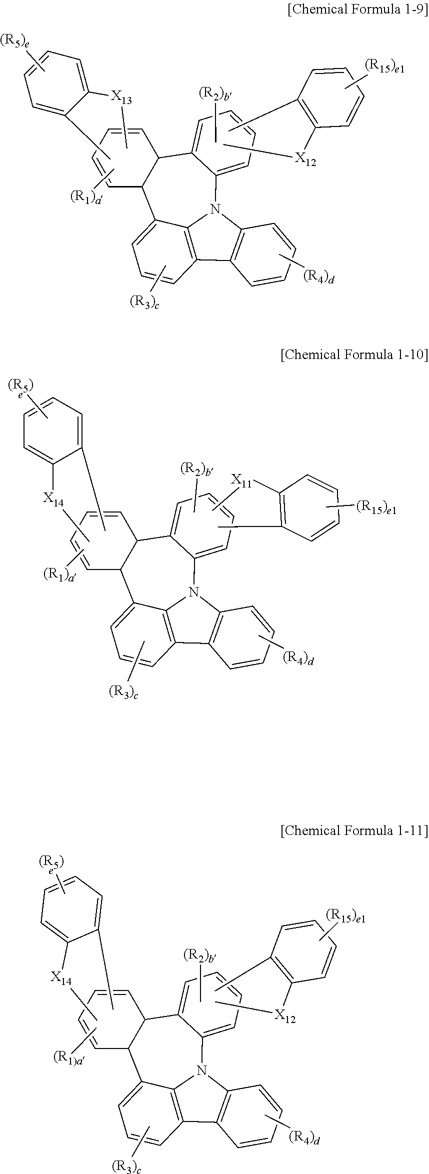

wherein in Chemical Formula 1-4 to Chemical Formula 1-11, X₁₁ to X₁₄, R₁ to R₅, R₁₅, a to e and e1 have the same definitions as above.

According to one embodiment of the present specification, X₁ is a direct bond.

According to one embodiment of the present specification, X₂ is a direct bond.

According to one embodiment of the present specification, X₁ is CRaRb.

According to one embodiment of the present specification, X₁ is O.

According to one embodiment of the present specification, X₁ is S.

According to one embodiment of the present specification, X₂ is CRaRb.

According to one embodiment of the present specification, X₂ is O.

According to one embodiment of the present specification, X₂ is S.

According to one embodiment of the present specification, X₁ is a direct bond, and X₂ is CRaRb.

According to one embodiment of the present specification, X₁ is a direct bond, and X₂ is O.

According to one embodiment of the present specification, X₁ is a direct bond, and X₂ is S.

According to one embodiment of the present specification, X₂ is a direct bond, and X₁ is CRaRb.

According to one embodiment of the present specification, X₂ is a direct bond, and X₁ is O.

According to one embodiment of the present specification, X₂ is a direct bond, and X₁ is S.

According to one embodiment of the present specification, X₁₁ is a direct bond.

According to one embodiment of the present specification, X₁₂ is a direct bond.

According to one embodiment of the present specification, X₁₁ is CRaRb.

According to one embodiment of the present specification, X₁₁ is O.

According to one embodiment of the present specification, X₁₁ is S.

According to one embodiment of the present specification, X₁₂ is CRaRb.

According to one embodiment of the present specification, X₁₂ is O.

According to one embodiment of the present specification, X₁₂ is S.

According to one embodiment of the present specification, X₁₁ is a direct bond, and X₁₂ is CRaRb.

According to one embodiment of the present specification, X₁₁ is a direct bond, and X₁₂ is O.

According to one embodiment of the present specification, X₁₁ is a direct bond, and X₁₂ is S.

According to one embodiment of the present specification, X₁₂ is a direct bond, and X₁₁ is CRaRb.

According to one embodiment of the present specification, X₁₂ is a direct bond, and X₁₁ is O.

According to one embodiment of the present specification, X₁₂ is a direct bond, and X₁₁ is S.

According to one embodiment of the present specification, X₁₃ is a direct bond.

According to one embodiment of the present specification, X₁₁ is a direct bond.

According to one embodiment of the present specification, X₁₃ is CRaRb.

According to one embodiment of the present specification, X₁₃ is O.

According to one embodiment of the present specification, X₁₃ is S.

According to one embodiment of the present specification, X₁₄ is CRaRb.

According to one embodiment of the present specification, X₁₄ is O.

According to one embodiment of the present specification, X₁₄ is S.

According to one embodiment of the present specification, X₁₃ is a direct bond, and X₁₄ is CRaRb.

According to one embodiment of the present specification, X₁₃ is a direct bond, and X₁₄ is O.

According to one embodiment of the present specification, X₁₃ is a direct bond, and X₁₄ is S.

According to one embodiment of the present specification, X₁₄ is a direct bond, and X₁₃ is CRaRb.

According to one embodiment of the present specification, X₁₄ is a direct bond, and X₁₃ is O.

According to one embodiment of the present specification, X₁₄ is a direct bond, and X₁₃ is S.

According to one embodiment of the present specification, in Chemical Formula 1-3, any one of X₁₁ to X₁₄ is a direct bond, and the rest are CRaRb, O or S.

According to one embodiment of the present specification, in Chemical Formula 1-3, any one of X₁₁ to X₁₄ is a direct bond, and any one of the rest is O or S.

According to one embodiment of the present specification, in Chemical Formula 1-3, any one of X₁₁ to X₁₄ is a direct bond, and any one of the rest is O.

According to one embodiment of the present specification, in Chemical Formula 1-3, any one of X₁₁ to X₁₄ is a direct bond, and any one of the rest is S.

According to one embodiment of the present specification, Z1 and Z2 are N.

According to one embodiment of the present specification, Z1 is N.

According to one embodiment of the present specification, Z2 is N.

According to one embodiment of the present specification, Z1 is CRc.

According to one embodiment of the present specification, Z2 is CRc.

According to one embodiment of the present specification, Y1 to Y3 are N.

According to one embodiment of the present specification, Y1 to Y3 are CRd.

According to one embodiment of the present specification, Y1 is N, and Y2 and Y3 are CRd.

According to one embodiment of the present specification, Y2 is N, and Y1 and Y3 are CRd.

According to one embodiment of the present specification, Y3 is N, and Y1 and Y2 are CRd.

According to one embodiment of the present specification, Y1 and Y2 are N, and Y3 is CRd.

According to one embodiment of the present specification, Y2 and Y3 are N, and Y1 is CRd.

According to one embodiment of the present specification, Y1 and Y3 are N, and Y2 is CRd.

According to one embodiment of the present specification, any one of R₁ to R₅ is Chemical Formula 3.

According to one embodiment of the present specification, R₁ is Chemical Formula 3.

According to one embodiment of the present specification, R₂ is Chemical Formula 3.

According to one embodiment of the present specification, R₃ is Chemical Formula 3.

According to one embodiment of the present specification, R₄ is Chemical Formula 3.

According to one embodiment of the present specification, R₅ is Chemical Formula 3.

According to one embodiment of the present specification, any two of R₁ to R₅ are Chemical Formula 3.

According to one embodiment of the present specification, R₁ and R₂ are Chemical Formula 3.

According to one embodiment of the present specification, R₂ and R₃ are Chemical Formula 3.

According to one embodiment of the present specification, R₂ and R₄ are Chemical Formula 3.

According to one embodiment of the present specification, R₂ and R₅ are Chemical Formula 3.

According to one embodiment of the present specification, R₃ and R₄ are Chemical Formula 3.

According to one embodiment of the present specification, R₃ and R₅ are Chemical Formula 3.

According to one embodiment of the present specification, R₄ and R₅ are Chemical Formula 3.

According to one embodiment of the present specification, the rest of R₁ to R₅ that are not Chemical Formula 3 are the same as or different from each other, and each independently is hydrogen, deuterium, a substituted or unsubstituted alkyl group, a substituted or unsubstituted aryl group, a substituted or unsubstituted heteroaryl group, or a substituted or unsubstituted cycloalkyl group.

According to one embodiment of the present specification, the rest of R₁ to R₅ that are not Chemical Formula 3 are hydrogen.

According to one embodiment of the present specification, L1 is a direct bond, a substituted or unsubstituted alkylene group, a substituted or unsubstituted arylene group, or a substituted or unsubstituted heteroarylene group.

According to one embodiment of the present specification, L1 is a direct bond, an alkylene group, an arylene group or a heteroarylene group.

According to one embodiment of the present specification, L1 is a direct bond, an alkylene group having 1 to 10 carbon atoms, an arylene group having 6 to 30 carbon atoms, or a heteroarylene group having 3 to 30 carbon atoms.

According to one embodiment of the present specification, L1 is a direct bond, an arylene group having 6 to 30 carbon atoms, or a heteroarylene group having 3 to 30 carbon atoms.

According to one embodiment of the present specification, L1 is a direct bond, a phenylene group, a divalent biphenyl group, a divalent terphenyl group, a divalent naphthyl group, a divalent anthracene group, a divalent phenanthrene group, a divalent triphenylene group, a divalent pyridine group, a divalent pyrimidine group, a divalent triazine group, a divalent carbazole group, a divalent dibenzofuran group, or a divalent dibenzothiophene group.

According to one embodiment of the present specification, L1 is a direct bond or a phenylene group.

According to one embodiment of the present specification, Ra and Rb are the same as or different from each other, and each independently is a substituted or unsubstituted alkyl group or a substituted or unsubstituted aryl group.

According to one embodiment of the present specification, Ra and Rb are the same as or different from each other, and each independently is a substituted or unsubstituted alkyl group having 1 to 10 carbon atoms.

According to one embodiment of the present specification, Ra and Rb are the same as or different from each other, and each independently is an alkyl group having 1 to 10 carbon atoms.

According to one embodiment of the present specification, Ra and Rb are the same as or different from each other, and each independently is a methyl group, an ethyl group, a propyl group, an isopropyl group, a butyl group, a tert-butyl group, an isobutyl group, a pentyl group or a hexyl group.

According to one embodiment of the present specification, Ra and Rb are a methyl group.

According to one embodiment of the present specification, Ra and Rb are the same as or different from each other, and each independently is a substituted or unsubstituted aryl group.

According to one embodiment of the present specification, Ra and Rb are the same as or different from each other, and each independently is a substituted or unsubstituted aryl group having 6 to 30 carbon atoms.

According to one embodiment of the present specification, Ra and Rb are the same as or different from each other, and each independently is an aryl group having 6 to 30 carbon atoms.

According to one embodiment of the present specification, Ra and Rb are the same as or different from each other, and each independently is a phenyl group, a biphenyl group, a terphenyl group, an anthracene group or a naphthyl group.

According to one embodiment of the present specification, Ra and Rb are a phenyl group.

According to one embodiment of the present specification, at least one of Y1 to Y3 is CRd, and Rd is hydrogen, a substituted or unsubstituted aryl group or a substituted or unsubstituted heteroaryl group.

According to one embodiment of the present specification, at least one of Y1 to Y3 is CRd, and Rd is hydrogen, a substituted or unsubstituted aryl group having 6 to 30 carbon atoms, or a substituted or unsubstituted heteroaryl group having 3 to 30 carbon atoms.

According to one embodiment of the present specification, at least one of Y1 to Y3 is CRd, and Rd is hydrogen, an aryl group having 6 to 20 carbon atoms or a heteroaryl group having 3 to 30 carbon atoms.

According to one embodiment of the present specification, at least one of Y1 to Y3 is CRd, and Rd is hydrogen, an aryl group having 6 to 15 carbon atoms or a heteroaryl group having 3 to 30 carbon atoms.

According to one embodiment of the present specification, at least one of Y1 to Y3 is CRd, and Rd is hydrogen, a phenyl group, a biphenyl group, a terphenyl group, a naphthyl group, an anthracene group, a phenanthrene group, a triphenylene group, a fluorene group, a pyridine group, a pyrimidine group, a triazine group, a carbazole group, a dibenzofuran group or a dibenzothiophene group.

According to one embodiment of the present specification, at least one of Y1 to Y3 is CRd, and Rd is hydrogen, a phenyl group, a biphenyl group or a pyridine group.

According to one embodiment of the present specification, Rd has adjacent substituents bonding to each other to form a substituted or unsubstituted ring.

According to one embodiment of the present specification, Rd has adjacent substituents bonding to each other to form a substituted or unsubstituted aromatic ring, a substituted or unsubstituted alicyclic ring or a substituted or unsubstituted heteroring.

According to one embodiment of the present specification, Rd has adjacent substituents bonding to each other to form an aromatic ring, an alicyclic ring or a heteroring.

According to one embodiment of the present specification, Rd has adjacent substituents bonding to each other to form an aromatic ring or a heteroring.

According to one embodiment of the present specification, Rd has adjacent substituents bonding to each other to form a benzene ring.

According to one embodiment of the present specification, at least one pair of Rd adjacent to each other bond to form a substituted or unsubstituted ring.

According to one embodiment of the present specification, at least one pair of Rd adjacent to each other bond to form a substituted or unsubstituted aromatic ring, a substituted or unsubstituted alicyclic ring or a substituted or unsubstituted heteroring.

According to one embodiment of the present specification, at least one pair of Rd adjacent to each other bond to form an aromatic ring or a heteroring.

According to one embodiment of the present specification, at least one pair of Rd adjacent to each other bond to form a benzene ring.

In one embodiment of the present specification, Chemical Formula 1 can be any one of the following compounds:

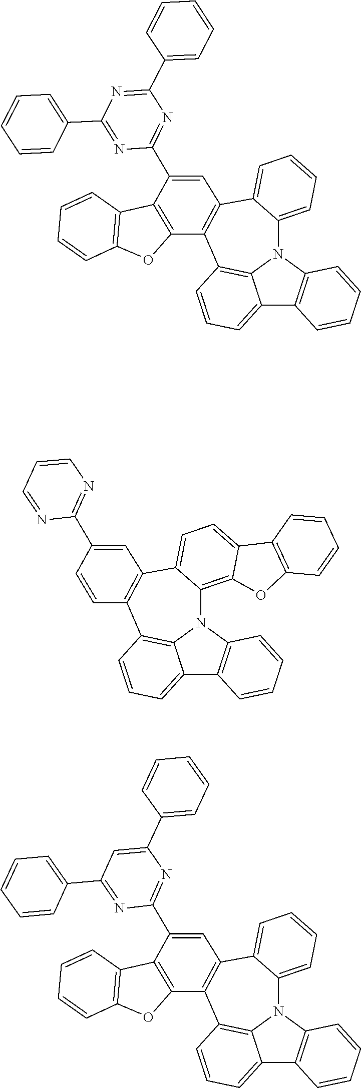 [SMILES: c1ccc(-c2nc(-c3ccccc3)nc(-c3cc4c(c5oc6ccccc6c35)-c3cccc5c6ccccc6n(c35)-c3ccccc3-4)n2)cc1 ; c1cnc(-c2ccc3c(c2)-c2ccc4c(oc5ccccc54)c2-n2c4ccccc4c4cccc-3c42)nc1 ; c1ccc(-c2cc(-c3ccccc3)nc(-c3cc4c(c5oc6ccccc6c35)-c3cccc5c6ccccc6n(c35)-c3ccccc3-4)n2)cc1]

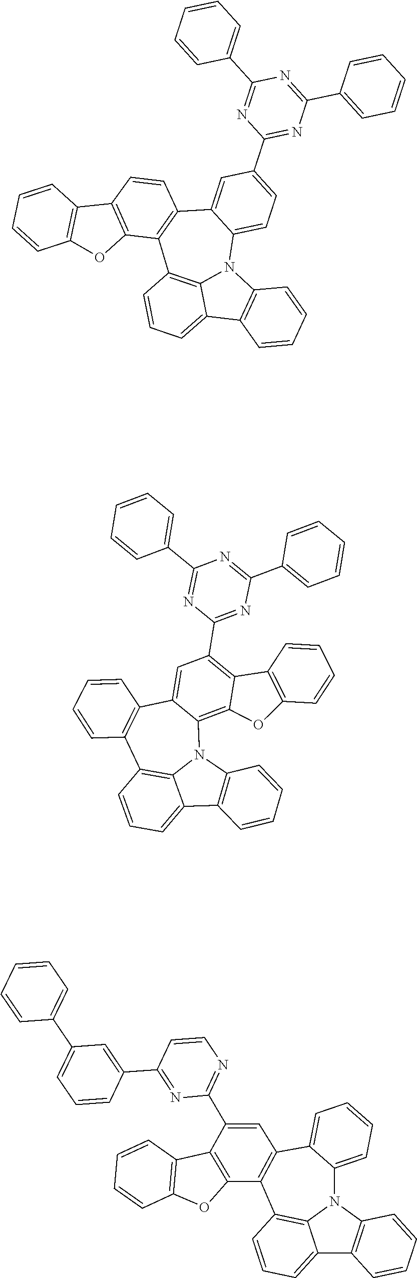 [SMILES: c1ccc(-c2nc(-c3ccccc3)nc(-c3ccc4c(c3)-c3ccc5c(oc6ccccc65)c3-c3cccc5c6ccccc6n-4c35)n2)cc1 ; c1ccc(-c2nc(-c3ccccc3)nc(-c3cc4c(c5oc6ccccc6c35)-n3c5ccccc5c5cccc(c53)-c3ccccc3-4)n2)cc1 ; c1ccc(-c2cccc(-c3ccnc(-c4cc5c(c6oc7ccccc7c46)-c4cccc6c7ccccc7n(c46)-c4ccccc4-5)n3)c2)cc1]

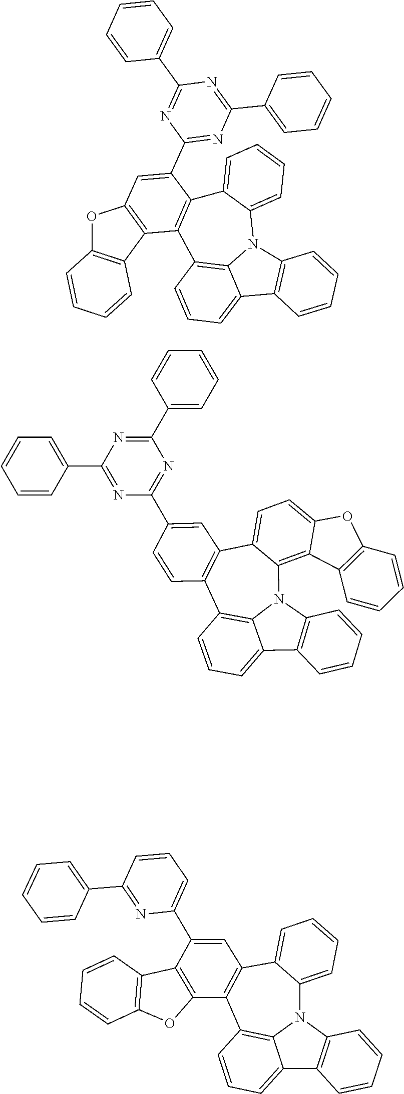

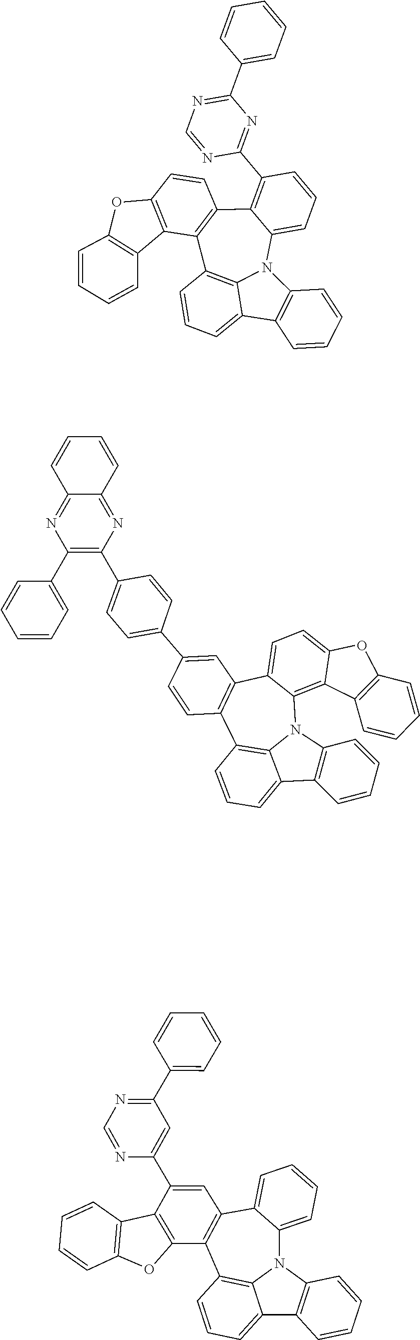 [SMILES: c1ccc(-c2ncnc(-c3cccc4c3-c3ccc5oc6ccccc6c5c3-c3cccc5c6ccccc6n-4c35)n2)cc1 ; c1ccc(-c2nc3ccccc3nc2-c2ccc(-c3ccc4c(c3)-c3ccc5oc6ccccc6c5c3-n3c5ccccc5c5cccc-4c53)cc2)cc1 ; c1ccc(-c2cc(-c3cc4c(c5oc6ccccc6c35)-c3cccc5c6ccccc6n(c35)-c3ccccc3-4)ncn2)cc1]

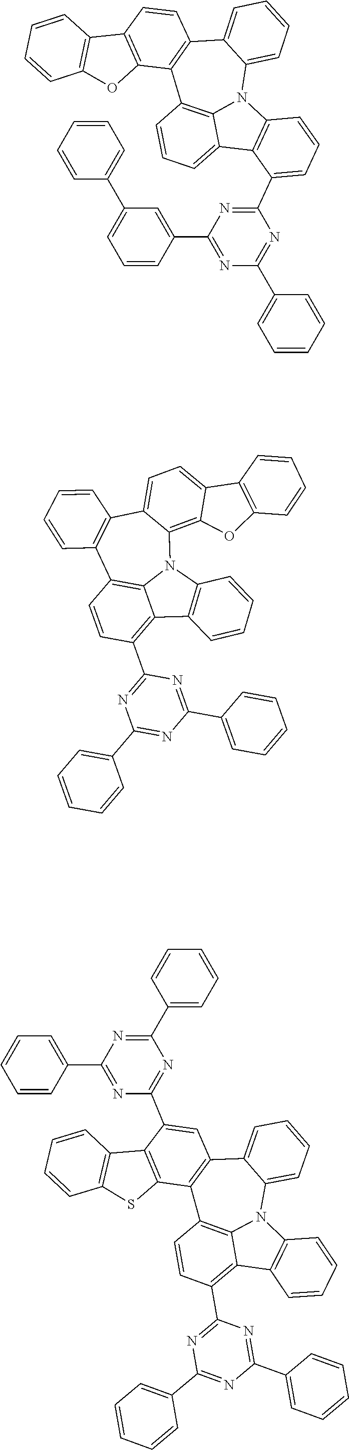 [SMILES: c1ccc(-c2cccc(-c3nc(-c4ccccc4)nc(-c4cccc5c4c4cccc6c4n5-c4ccccc4-c4ccc5c(oc7ccccc75)c4-6)n3)c2)cc1 ; c1ccc(-c2nc(-c3ccccc3)nc(-c3ccc4c5c3c3ccccc3n5-c3c(ccc5c3oc3ccccc35)-c3ccccc3-4)n2)cc1 ; c1ccc(-c2nc(-c3ccccc3)nc(-c3cc4c(c5sc6ccccc6c35)-c3ccc(-c5nc(-c6ccccc6)nc(-c6ccccc6)n5)c5c6ccccc6n(c35)-c3ccccc3-4)n2)cc1]

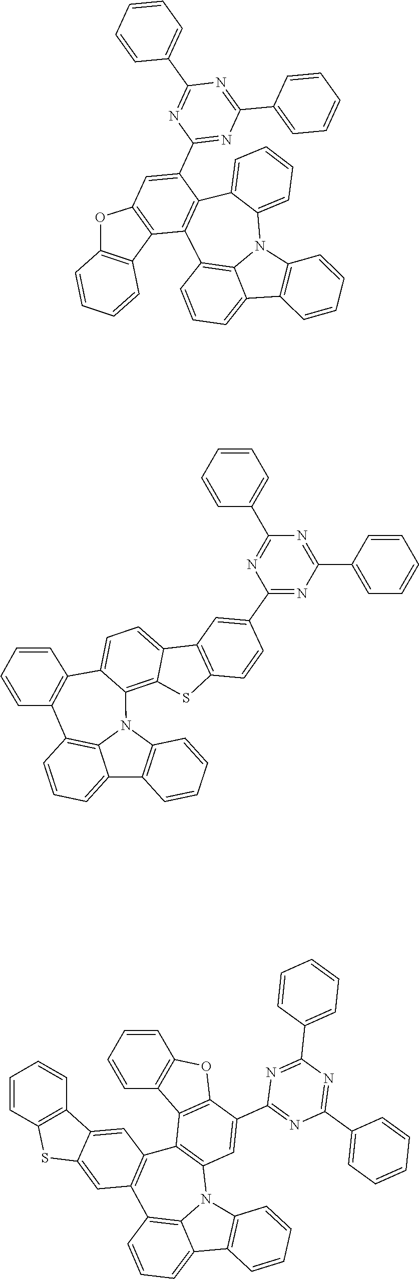 [SMILES: c1ccc(-c2nc(-c3ccccc3)nc(-c3cc4oc5ccccc5c4c4c3-c3ccccc3-n3c5ccccc5c5cccc-4c53)n2)cc1 ; c1ccc(-c2nc(-c3ccccc3)nc(-c3ccc4sc5c6c(ccc5c4c3)-c3ccccc3-c3cccc4c5ccccc5n-6c34)n2)cc1 ; c1ccc(-c2nc(-c3ccccc3)nc(-c3cc4c(c5c3oc3ccccc35)-c3cc5c(cc3-c3cccc6c7ccccc7n-4c36)sc3ccccc35)n2)cc1]

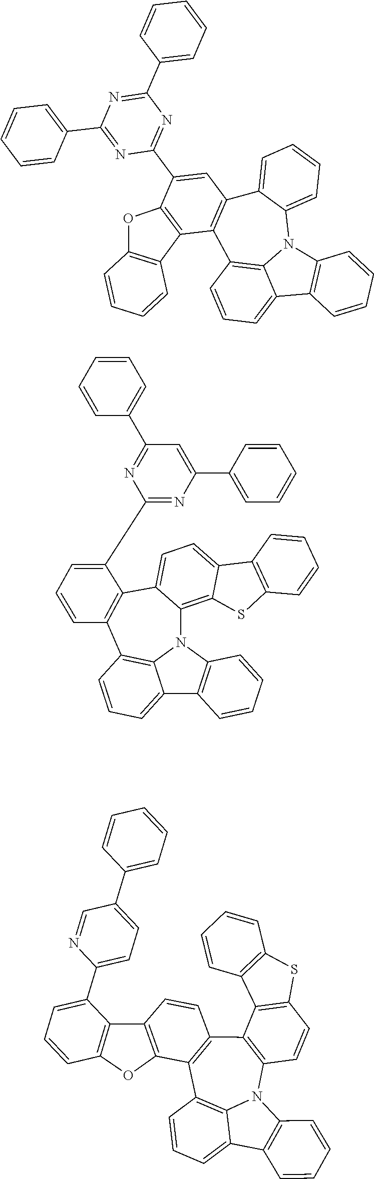

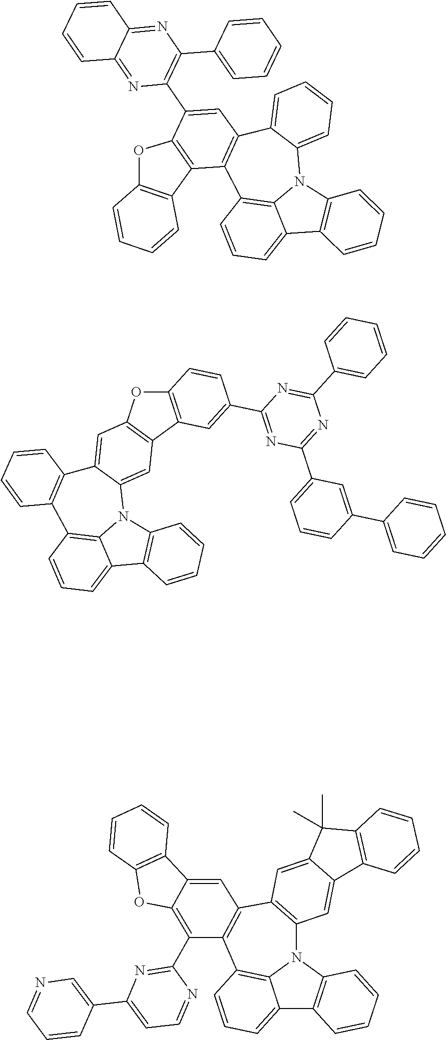 [SMILES: c1ccc(-c2nc3ccccc3nc2-c2cc3c(c4c2oc2ccccc24)-c2cccc4c5ccccc5n(c24)-c2ccccc2-3)cc1 ; c1ccc(-c2cccc(-c3nc(-c4ccccc4)nc(-c4ccc5oc6cc7c(cc6c5c4)-n4c5ccccc5c5cccc(c54)-c4ccccc4-7)n3)c2)cc1 ; CC1(C)c2ccccc2-c2cc3c(cc21)-c1cc2c(oc4ccccc42)c(-c2nccc(-c4cccnc4)n2)c1-c1cccc2c4ccccc4n-3c12]

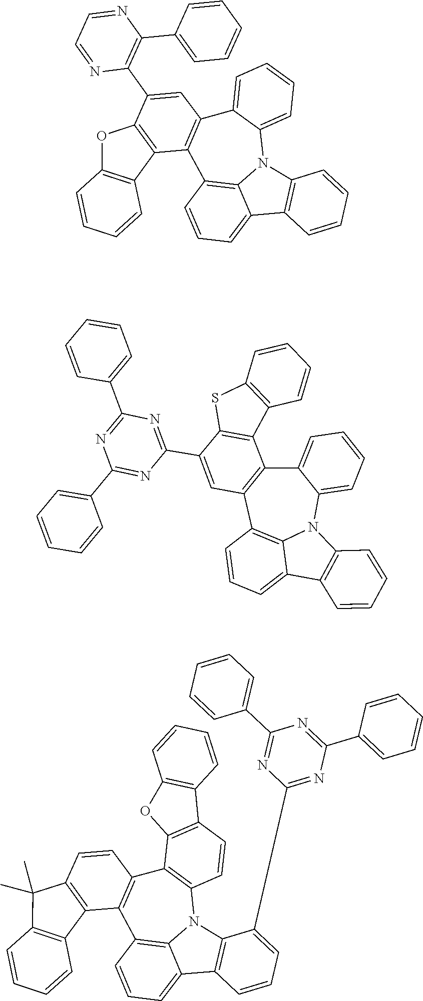 [SMILES: c1ccc(-c2nccnc2-c2cc3c(c4c2oc2ccccc24)-c2cccc4c5ccccc5n(c24)-c2ccccc2-3)cc1 ; c1ccc(-c2nc(-c3ccccc3)nc(-c3cc4c(c5c3sc3ccccc35)-c3ccccc3-n3c5ccccc5c5cccc-4c53)n2)cc1 ; CC1(C)c2ccccc2-c2c1ccc1c2-c2cccc3c4cccc(-c5nc(-c6ccccc6)nc(-c6ccccc6)n5)c4n(c23)-c2ccc3c(oc4ccccc43)c2-1]

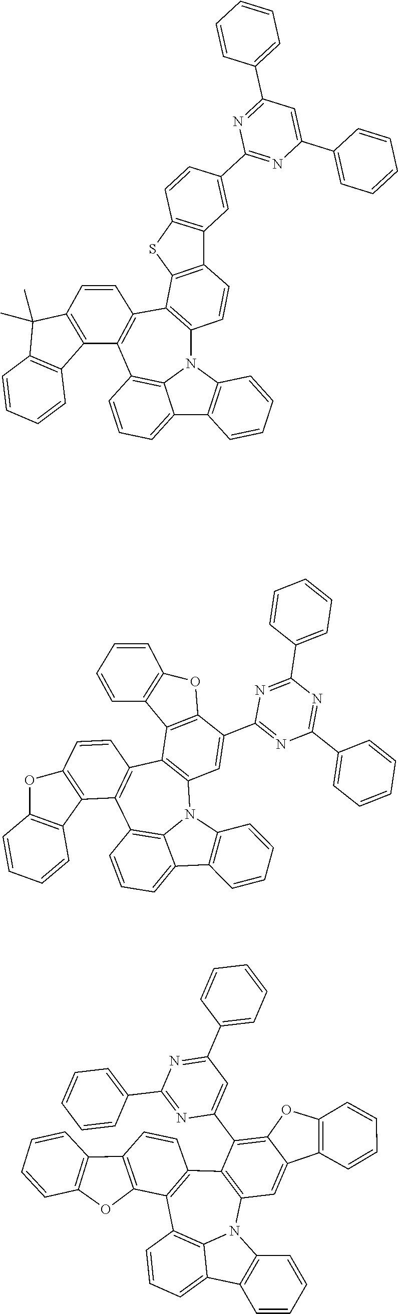 [SMILES: CC1(C)c2ccccc2-c2c1ccc1c2-c2cccc3c4ccccc4n(c23)-c2ccc3c(sc4ccc(-c5nc(-c6ccccc6)cc(-c6ccccc6)n5)cc43)c2-1 ; c1ccc(-c2nc(-c3ccccc3)nc(-c3cc4c(c5c3oc3ccccc35)-c3ccc5oc6ccccc6c5c3-c3cccc5c6ccccc6n-4c35)n2)cc1 ; c1ccc(-c2cc(-c3c4c(cc5c3oc3ccccc35)-n3c5ccccc5c5cccc(c53)-c3c-4ccc4c3oc3ccccc34)nc(-c3ccccc3)n2)cc1]

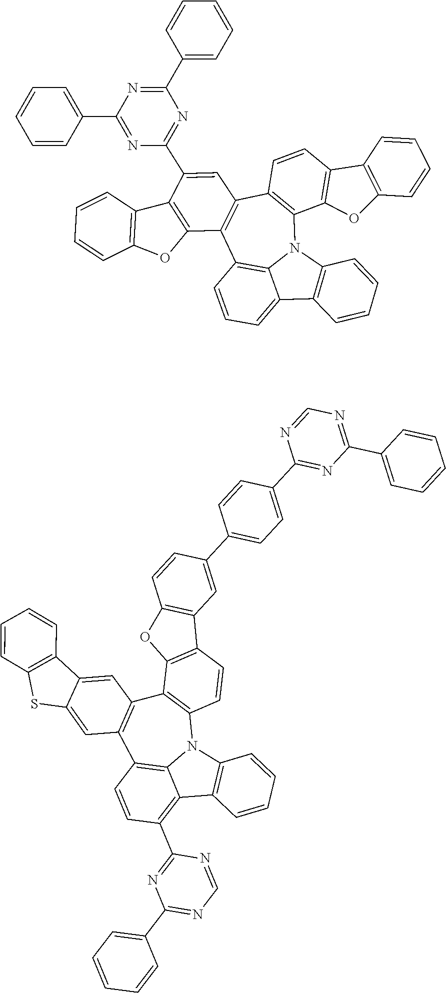 [SMILES: c1ccc(-c2nc(-c3ccccc3)nc(-c3cc4c(c5oc6ccccc6c35)-c3cccc5c6ccccc6n(c35)-c3c-4ccc4c3oc3ccccc34)n2)cc1 ; c1ccc(-c2ncnc(-c3ccc(-c4ccc5oc6c7c(ccc6c5c4)-n4c5ccccc5c5c(-c6ncnc(-c8ccccc8)n6)ccc(c54)-c4cc5sc6ccccc6c5cc4-7)cc3)n2)cc1]

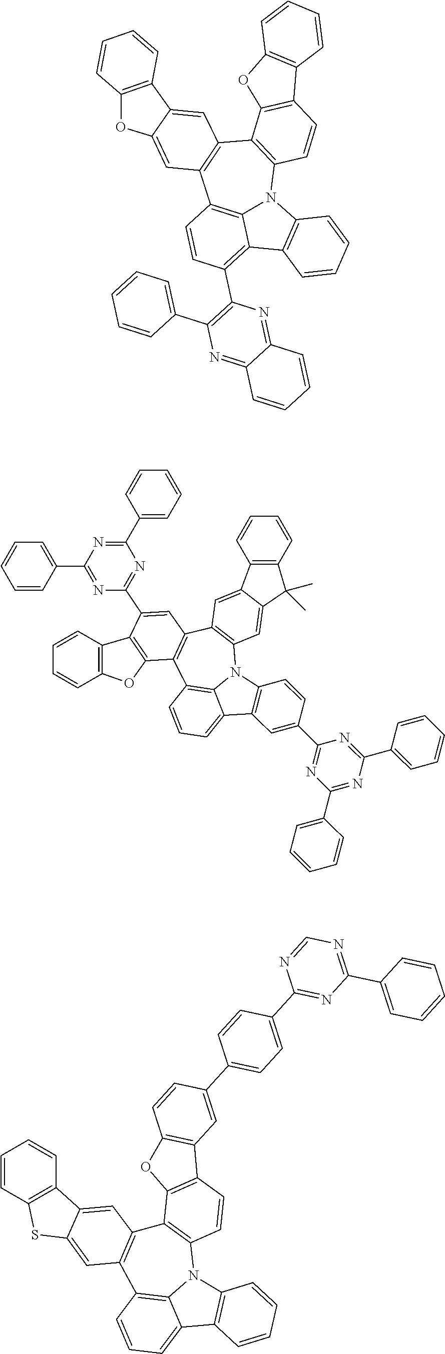 [SMILES: c1ccc(-c2nc3ccccc3nc2-c2ccc3c4c2c2ccccc2n4-c2ccc4c(oc5ccccc54)c2-c2cc4c(cc2-3)oc2ccccc24)cc1 ; CC1(C)c2ccccc2-c2cc3c(cc21)-n1c2ccc(-c4nc(-c5ccccc5)nc(-c5ccccc5)n4)cc2c2cccc(c21)-c1c-3cc(-c2nc(-c3ccccc3)nc(-c3ccccc3)n2)c2c1oc1ccccc12 ; c1ccc(-c2ncnc(-c3ccc(-c4ccc5oc6c7c(ccc6c5c4)-n4c5ccccc5c5cccc(c54)-c4cc5sc6ccccc6c5cc4-7)cc3)n2)cc1]

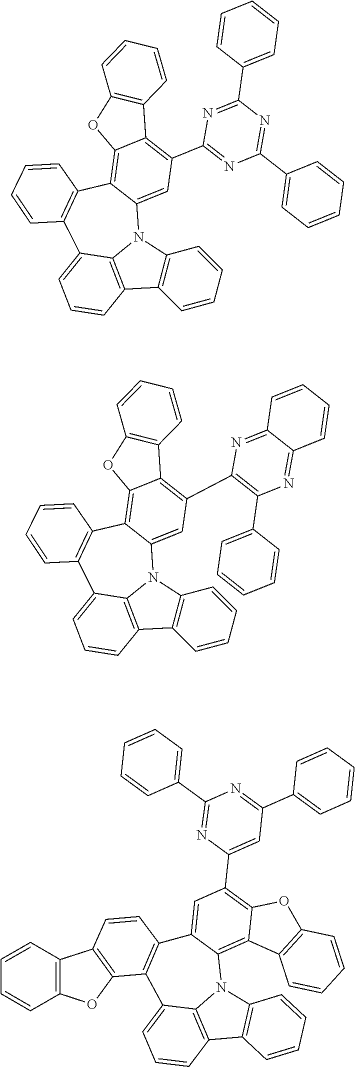 [SMILES: c1ccc(-c2nc(-c3ccccc3)nc(-c3cc4c(c5oc6ccccc6c35)-c3ccccc3-c3cccc5c6ccccc6n-4c35)n2)cc1 ; c1ccc(-c2nc3ccccc3nc2-c2cc3c(c4oc5ccccc5c24)-c2ccccc2-c2cccc4c5ccccc5n-3c24)cc1 ; c1ccc(-c2cc(-c3cc4c(c5c3oc3ccccc35)-n3c5ccccc5c5cccc(c53)-c3c-4ccc4c3oc3ccccc34)nc(-c3ccccc3)n2)cc1]

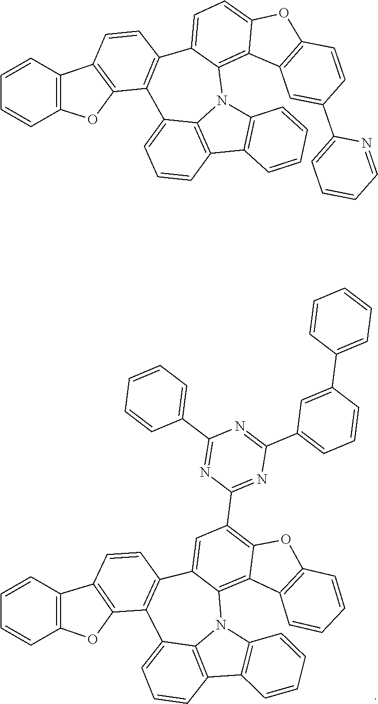 [SMILES: c1ccc(-c2ccc3oc4ccc5c(c4c3c2)-n2c3ccccc3c3cccc(c32)-c2c-5ccc3c2oc2ccccc23)nc1 ; c1ccc(-c2cccc(-c3nc(-c4ccccc4)nc(-c4cc5c(c6c4oc4ccccc46)-n4c6ccccc6c6cccc(c64)-c4c-5ccc5c4oc4ccccc45)n3)c2)cc1]

In the compound of Chemical Formula 1, substituents can bond using methods known in the art, and types, positions or the number of the substituents can vary depending on technologies known in the art.

A conjugation length of the heterocyclic compound and an energy band gap thereof are closely related. Specifically, as a conjugation length of the compound increases, an energy band gap thereof decreases.

By introducing various substituents to the core structure as above, compounds having various energy band gaps can be synthesized in the present disclosure. In addition, by introducing various substituents to the core structure having structures as above, HOMO and LUMO energy levels of the compound can also be controlled in the present disclosure.

In addition, by introducing various substituents to the core structure having structures as above, compounds having unique properties of the introduced substituents can be synthesized. For example, by introducing substituents normally used as a hole injection layer material, a material for hole transfer, a light emitting layer material and an electron transfer layer material used for manufacturing an organic light emitting device to the core structure, materials satisfying needs required from each organic material layer can be synthesized.

In addition, an organic light emitting device according to the present disclosure includes a first electrode; a second electrode provided opposite to the first electrode; and one or more organic material layers provided between the first electrode and the second electrode, wherein one or more layers of the organic material layers include the heterocyclic compound described above.

The organic light emitting device of the present disclosure can be manufactured using common organic light emitting device manufacturing methods and materials except that one or more organic material layers are formed using the heterocyclic compound described above.

The heterocyclic compound can be formed into an organic material layer through a solution coating method as well as a vacuum deposition method when manufacturing the organic light emitting device. Herein, the solution coating method means spin coating, dip coating, inkjet printing, screen printing, a spray method, roll coating and the like, but is not limited thereto.

The organic material layer of the organic light emitting device of the present disclosure can be formed in a single layer structure, but can be formed in a multilayer structure in which two or more organic material layers are laminated. For example, the organic light emitting device of the present disclosure can have a structure including a hole injection layer, a hole transfer layer, a layer carrying out hole injection and hole transfer at the same time, a light emitting layer, an electron transfer layer, an electron injection layer and the like as the organic material layer. However, the structure of the organic light emitting device is not limited thereto, and can include a smaller number of organic material layers or a larger number of organic material layers.

In the organic light emitting device of the present disclosure, the organic material layer can include one or more of an electron transfer layer, an electron injection layer, and a layer carrying out electron injection and electron transfer at the same time, and one or more of the above-described layers can include the heterocyclic compound of Chemical Formula 1.

In another organic light emitting device, the organic material layer can include an electron transfer layer or an electron injection layer, and the electron transfer layer or the electron injection layer can include the heterocyclic compound of Chemical Formula 1.

In the organic light emitting device of the present disclosure, the organic material layer can include one or more of a hole injection layer, a hole transfer layer, and a layer carrying out hole injection and hole transfer at the same time, and one or more of the above-described layers can include the heterocyclic compound of Chemical Formula 1.

In another organic light emitting device, the organic material layer can include a hole injection layer or a hole transfer layer, and the hole transfer layer or the hole injection layer can include the heterocyclic compound of Chemical Formula 1.

In another embodiment, the organic material layer includes a light emitting layer, and the light emitting layer includes the heterocyclic compound of Chemical Formula 1. As one example, the heterocyclic compound of Chemical Formula 1 can be included as a host of the light emitting layer.

In one embodiment of the present specification, the organic light emitting device is a green organic light emitting device in which the light emitting layer includes the heterocyclic compound of Chemical Formula 1 as a host.

According to one embodiment of the present specification, the organic light emitting device is a red organic light emitting device in which the light emitting layer includes the heterocyclic compound of Chemical Formula 1 as a host.

In another embodiment, the organic light emitting device is a blue organic light emitting device in which the light emitting layer includes the heterocyclic compound of Chemical Formula 1 as a host.

As another example, the light emitting layer including the heterocyclic compound of Chemical Formula 1 includes the heterocyclic compound of Chemical Formula 1 as a host.

As another example, the light emitting layer including the heterocyclic compound of Chemical Formula 1 includes the heterocyclic compound of Chemical Formula 1 as a green host.

As another example, the light emitting layer including the heterocyclic compound of Chemical Formula 1 includes the heterocyclic compound of Chemical Formula 1 as a host, and can include a fluorescent host or a phosphorescent dopant.

In another embodiment, the light emitting layer including the heterocyclic compound of Chemical Formula 1 includes the heterocyclic compound of Chemical Formula 1 as a host, includes a fluorescent dopant or a phosphorescent dopant, and can include other organic compounds, metals or metal compounds as a dopant.

In one embodiment of the present specification, the light emitting layer includes a dopant.

As another example, the light emitting layer includes the heterocyclic compound of Chemical Formula 1 as a host, and a phosphorescent dopant can be used therewith.

As another example, the light emitting layer includes the heterocyclic compound of Chemical Formula 1 as a green host, and a phosphorescent dopant can be used therewith.

As another example, the light emitting layer includes the heterocyclic compound of Chemical Formula 1 as a host, and an iridium (Ir)-based dopant can be used therewith.

As another example, the light emitting layer includes the heterocyclic compound of Chemical Formula 1 as a green host, and an iridium (Ir)-based dopant can be used therewith.

In one embodiment of the present specification, the light emitting layer includes a phosphorescent host and a phosphorescent dopant.

In one embodiment of the present specification, the light emitting layer includes a green phosphorescent host and a green phosphorescent dopant.

In one embodiment of the present specification, the light emitting layer includes a host and a dopant in a mass ratio of 99:1 to 70:30.

In one embodiment of the present specification, the light emitting layer includes a host and a dopant in a mass ratio of 99:1 to 75:25.

In one embodiment of the present specification, the light emitting layer includes a host and a dopant in a mass ratio of 95:5 to 80:20.

In one embodiment of the present specification, the first electrode is an anode, and the second electrode is a cathode.

According to another embodiment, the first electrode is a cathode, and the second electrode is an anode.

(1) Anode/hole transfer layer/light emitting layer/cathode

(2) Anode/hole injection layer/hole transfer layer/light emitting layer/cathode

(3) Anode/hole injection layer/hole buffer layer/hole transfer layer/light emitting layer/cathode

(4) Anode/hole transfer layer/light emitting layer/electron transfer layer/cathode

(5) Anode/hole transfer layer/light emitting layer/electron transfer layer/electron injection layer/cathode

(6) Anode/hole injection layer/hole transfer layer/light emitting layer/electron transfer layer/cathode

(7) Anode/hole injection layer/hole transfer layer/light emitting layer/electron transfer layer/electron injection layer/cathode

(8) Anode/hole injection layer/hole buffer layer/hole transfer layer/light emitting layer/electron transfer layer/cathode

(9) Anode/hole injection layer/hole buffer layer/hole transfer layer/light emitting layer/electron transfer layer/electron injection layer/cathode

(10) Anode/hole transfer layer/electron blocking layer/light emitting layer/electron transfer layer/cathode

(11) Anode/hole transfer layer/electron blocking layer/light emitting layer/electron transfer layer/electron injection layer/cathode

(12) Anode/hole injection layer/hole transfer layer/electron blocking layer/light emitting layer/electron transfer layer/cathode

(13) Anode/hole injection layer/hole transfer layer/electron blocking layer/light emitting layer/electron transfer layer/electron injection layer/cathode

(14) Anode/hole transfer layer/light emitting layer/hole blocking layer/electron transfer layer/cathode

(15) Anode/hole transfer layer/light emitting layer/hole blocking layer/electron transfer layer/electron injection layer/cathode

(16) Anode/hole injection layer/hole transfer layer/light emitting layer/hole blocking layer/electron transfer layer/cathode

(17) Anode/hole injection layer/hole transfer layer/light emitting layer/hole blocking layer/electron transfer layer/electron injection layer/cathode

(18) Anode/hole injection layer/hole transfer layer/electron blocking layer/light emitting layer/hole blocking layer/electron injection and transfer layer/cathode

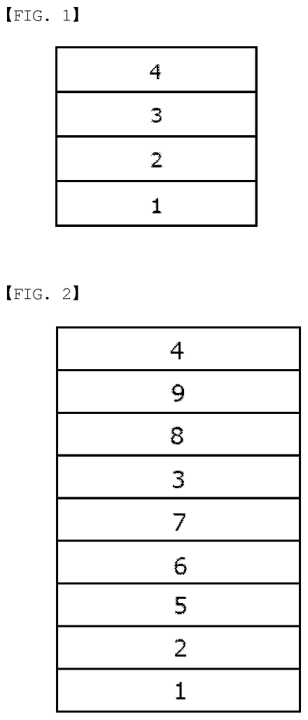

The organic light emitting device of the present disclosure can have structures as illustrated in FIG. 1 to FIG. 3, however, the structure is not limited thereto.

FIG. 1 illustrates a structure of the organic light emitting device in which a first electrode (2), a light emitting layer (3) and a second electrode (4) are consecutively laminated on a substrate (1). In such a structure, the heterocyclic compound of Chemical Formula 1 can be included in the light emitting layer (3).

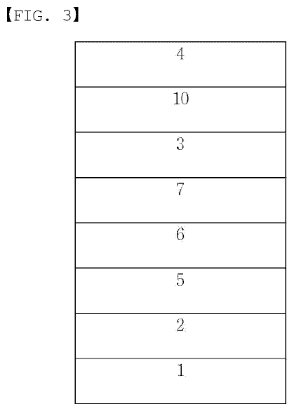

FIG. 2 illustrates a structure of the organic light emitting device in which a first electrode (2), a hole injection layer (5), a hole transfer layer (6), an electron blocking layer (7), a light emitting layer (3), an electron transfer layer (8), an electron injection layer (9) and a second electrode (4) are consecutively laminated on a substrate (1). In such a structure, the heterocyclic compound of Chemical Formula 1 can be included in the light emitting layer (3).

FIG. 3 illustrates a structure of the organic light emitting device in which a first electrode (2), a hole injection layer (5), a hole transfer layer (6), an electron blocking layer (7), a light emitting layer (3), an electron injection and transfer layer (10) and a second electrode (4) are consecutively laminated on a substrate (1). In such a structure, the heterocyclic compound of Chemical Formula 1 can be included in the light emitting layer (3).

For example, the organic light emitting device according to the present disclosure can be manufactured by forming an anode on a substrate by depositing a metal, a metal oxide having conductivity, or an alloy thereof using a physical vapor deposition (PVD) method such as sputtering or e-beam evaporation, forming an organic material layer including one or more layers selected from the group consisting of a hole injection layer, a hole transfer layer, a layer carrying out hole transfer and hole injection at the same time, a light emitting layer, an electron transfer layer, an electron injection layer, and a layer carrying out electron transfer and electron injection at the same time thereon, and then depositing a material usable as a cathode thereon. In addition to such a method, the organic light emitting device can also be manufactured by consecutively depositing a cathode material, an organic material layer and an anode material on a substrate.

The organic material layer can have a multilayer structure including a hole injection layer, a hole transfer layer, a light emitting layer, an electron transfer layer and the like, however, the structure is not limited thereto, and the organic material layer can have a single layer structure. In addition, the organic material layer can be prepared to have a smaller number of layers through a solvent process such as spin coating, dip coating, doctor blading, screen printing, inkjet printing or a thermal transfer method instead of a deposition method using various polymer materials.

The anode is an electrode injecting holes, and as the anode material, materials having large work function are normally preferred so that hole injection to an organic material layer is smooth. Specific examples of the anode material that can be used in the present disclosure include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene] (PEDOT), polypyrrole and polyaniline, and the like, but are not limited thereto.

The cathode is an electrode injecting electrons, and as the cathode material, materials having small work function are normally preferred so that electron injection to an organic material layer is smooth. Specific examples of the cathode material include metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

The hole injection layer is a layer performing a role of smoothly injecting holes from an anode to a light emitting layer, and the hole injection material is a material capable of favorably receiving holes from an anode at a low voltage. The highest occupied molecular orbital (HOMO) of the hole injection material is preferably in between the work function of an anode material and the HOMO of surrounding organic material layers. Specific examples of the hole injection material include metal porphyrins, oligothiophene, arylamine-based organic materials, hexanitrile hexaazatriphenylene-based organic materials, quinacridone-based organic materials, perylene-based organic materials, anthraquinone, and polyaniline- and polythiophene-based conductive polymers, and the like, but are not limited thereto. The hole injection layer can have a thickness of 1 nm to 150 nm. The hole injection layer having a thickness of 1 nm or greater has an advantage of preventing hole injection properties from declining, and the thickness being 150 nm or less has an advantage of preventing a driving voltage from increasing to enhance hole migration caused by the hole injection layer being too thick.

The hole transfer layer can perform a role of smoothly transferring holes. As the hole transfer material, materials capable of receiving holes from an anode or a hole injection layer, moving the holes to a light emitting layer, and having high mobility for the holes are suited. Specific examples thereof include arylamine-based organic materials, conductive polymers, block copolymers having conjugated parts and non-conjugated parts together, and the like, but are not limited thereto.

A hole buffer layer can be additionally provided between the hole injection layer and the hole transfer layer, and can include hole injection or transfer materials known in the art.

An electron blocking layer can be provided between the hole transfer layer and a light emitting layer. As the electron blocking layer, the spiro compound described above or materials known in the art can be used.

The light emitting layer can emit red, green or blue, and can be formed with a phosphorescence material or a fluorescence material. The light emitting material is a material capable of emitting light in a visible region by receiving holes and electrons from a hole transfer layer and an electron transfer layer, respectively, and binding the holes and the electrons, and is preferably a material having favorable quantum efficiency for fluorescence or phosphorescence. Specific examples thereof include 8-hydroxy-quinoline aluminum complexes (Alq₃); carbazole-based compounds; dimerized styryl compounds; BAlq; 10-hydroxybenzoquinoline-metal compounds; benzoxazole-, benzothiazole- and benzimidazole-based compounds; poly(p-phenylenevinylene) (PPV)-based polymers; spiro compounds; polyfluorene, rubrene, and the like, but are not limited thereto.

A host material of the light emitting layer can include fused aromatic ring derivatives, heteroring-containing compounds or the like. Specifically, as the fused aromatic ring derivative, anthracene derivatives, pyrene derivatives, naphthalene derivatives, pentacene derivatives, phenanthrene compounds, fluoranthene compounds and the like can be included, and as the heteroring-containing compound, carbazole derivatives, dibenzofuran derivatives, ladder-type furan compounds, pyrimidine derivatives and the like can be included, however, the host material is not limited thereto.

When the light emitting layer emits red light, phosphorescence materials such as PIQIr(acac) (bis(1-phenylisoquinoline)acetylacetonate iridium), PQIr(acac) (bis(1-phenylquinoline)acetylacetonate iridium), PQIr (tris(1-phenylquinoline)iridium) or PtOEP (octaethylporphyrin platinum), or fluorescence materials such as Alq₃ (tris(8-hydroxyquinolino)aluminum) can be used as the light emitting dopant, however, the light emitting dopant is not limited thereto. When the light emitting layer emits green light, phosphorescence materials such as Ir(ppy)₃ (fac tris(2-phenylpyridine)iridium), or fluorescence materials such as Alq₃ (tris(8-hydroxyquinolino)aluminum) can be used as the light emitting dopant, however, the light emitting dopant is not limited thereto. When the light emitting layer emits blue light, phosphorescence materials such as (4,6-F₂ppy)₂Irpic, or fluorescence materials such as spiro-DPVBi, spiro-6P, distyrylbenzene (DSB), distyrylarylene (DSA), PFO-based polymers or PPV-based polymers can be used as the light emitting dopant, however, the light emitting dopant is not limited thereto.

A hole blocking layer can be provided between an electron transfer layer and the light emitting layer, and materials known in the art can be used.

The electron transfer layer can perform a role of smoothly transferring electrons. As the electron transfer material, materials capable of favorably receiving electrons from a cathode, moving the electrons to a light emitting layer, and having high mobility for the electrons are suited. Specific examples thereof include Al complexes of 8-hydroxyquinoline; complexes including Alq₃; organic radical compounds; hydroxyflavon-metal complexes, and the like, but are not limited thereto. The electron transfer layer can have a thickness of 1 nm to 50 nm. The electron transfer layer having a thickness of 1 nm or greater has an advantage of preventing electron transfer properties from declining, and the thickness being 50 nm or less has an advantage of preventing a driving voltage from increasing to enhance electron migration caused by the electron transfer layer being too thick.

The electron injection layer can perform a role of smoothly injecting electrons. As the electron injection material, compounds having an electron transferring ability, having an electron injection effect from a cathode, having an excellent electron injection effect for a light emitting layer or light emitting material, and preventing excitons generated in the light emitting layer from moving to a hole injection layer, and, in addition thereto, having an excellent thin film forming ability are preferred. Specific examples thereof can include fluorenone, anthraquinodimethane, diphenoquinone, thiopyran dioxide, oxazole, oxadiazole, triazole, imidazole, perylene tetracarboxylic acid, fluorenylidene methane, anthrone or the like, and derivatives thereof, metal complex compounds, nitrogen-containing 5-membered ring derivatives, and the like, but are not limited thereto.

The metal complex compound includes 8-hydroxyquinolinato lithium, bis(8-hydroxyquinolinato)zinc, bis(8-hydroxyquinolinato)copper, bis(8-hydroxyquinolinato)-manganese, tris(8-hydroxyquinolinato)aluminum, tris(2-methyl-8-hydroxyquinolinato)aluminum, tris(8-hydroxy-quinolinato)gallium, bis(10-hydroxybenzo[h]quinolinato)-beryllium, bis(10-hydroxybenzo[h]quinolinato)zinc, bis(2-methyl-8-quinolinato)chlorogallium, bis(2-methyl-8-quinolinato) (o-cresolato)gallium, bis(2-methyl-8-quinolinato) (1-naphtholato)aluminum, bis(2-methyl-8-quinolinato) (2-naphtholato) gallium and the like, but is not limited thereto.

The hole blocking layer is a layer blocking holes from reaching a cathode, and can be generally formed under the same condition as the hole injection layer. Specific examples thereof can include oxadiazole derivatives, triazole derivatives, phenanthroline derivatives, BCP, aluminum complexes and the like, but are not limited thereto.

The organic light emitting device according to the present disclosure can be a top-emission type, a bottom-emission type or a dual-emission type depending on the materials used.

The organic light emitting device according to the present specification can be included and used in various electronic devices. For example, the electronic device can be a display panel, a touch panel, a solar module, a lighting device or the like, and is not limited thereto.

### EXAMPLES

The organic light emitting device of the present disclosure can be manufactured using common organic light emitting device manufacturing methods and materials except that one or more organic material layers are formed using the heterocyclic compound described above.

Methods for preparing the compound of Chemical Formula 1 and manufacturing an organic light emitting device using the same will be specifically described in the following examples. However, the following examples are for illustrative purposes only, and the scope of the present disclosure is not limited thereto.

In the following reaction formulae, as for types and the number of substituents, various types of intermediates can be synthesized by those skilled in the art properly selecting known starting materials. As the reaction type and reaction condition, those known in the art can be used.

### SYNTHESIS EXAMPLE

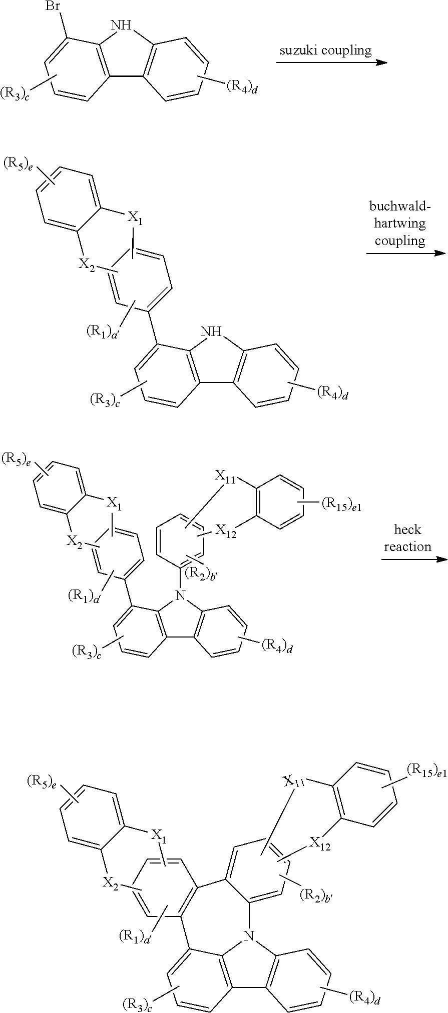

Substituents in the formula have the same definitions as in Chemical Formula 1-3.

The compounds of Chemical Formula 1 described in the present specification can all be prepared when properly combining preparation formulae described in the examples of the present specification with the intermediates based on common technical ideas.

### PREPARATION EXAMPLE

### [Preparation Example 1] Preparation of Compound 1-1

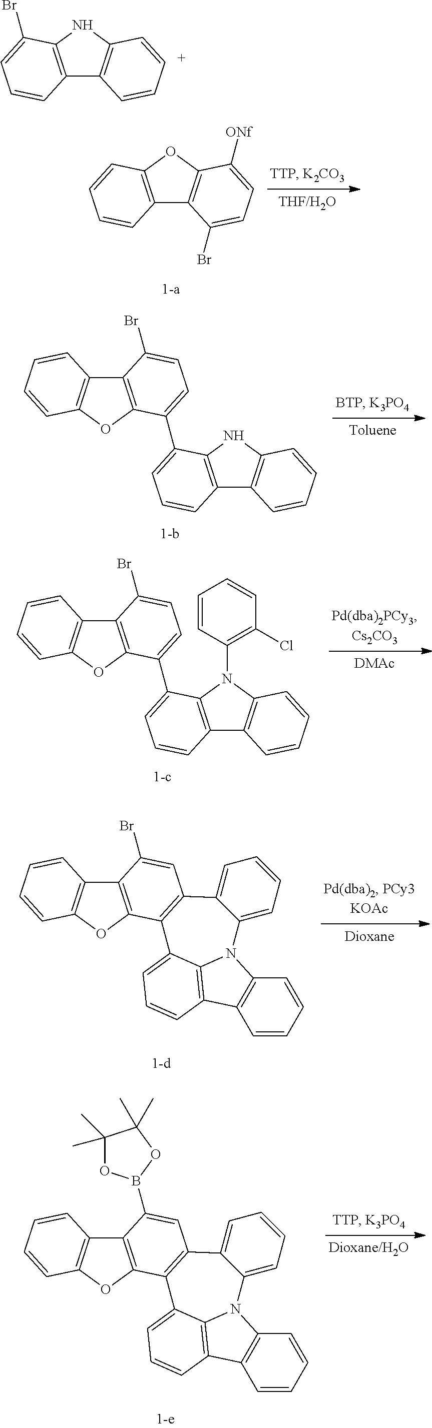

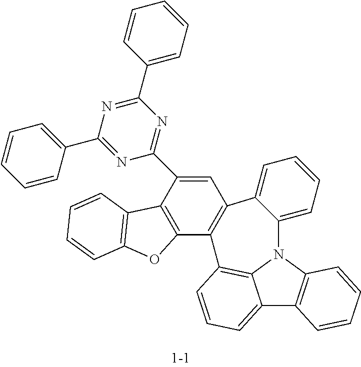 [SMILES: c1ccc(-c2nc(-c3ccccc3)nc(-c3cc4c(c5oc6ccccc6c35)-c3cccc5c6ccccc6n(c35)-c3ccccc3-4)n2)cc1]

1) Preparation of Compound 1-b

After dissolving 1-bromocarbazole (20.0 g, 81.26 mmol, 1.0 eq.), Compound 1-a (44.3 g, 81.26 mmol, 1.0 eq.) and Pd(PPh₃)₄ (0.94 g, 0.81 mmol, 0.01 eq.) in tetrahydrofuran (THF) (200 mL) and stirring the mixture, K₂CO₃ (22.5 g, 162.53 mmol, 2.0 eq.) dissolved in water (50 mL) was added thereto, and the result was stirred under reflux. When the reaction was finished, EA (ethyl acetate) was introduced thereto, and after separating the organic layer, the solvent was removed under vacuum. The product was completely dissolved in chloroform (CHCl₃), then washed with water, and the solvent was vacuum concentrated by about 80%. Ethanol was introduced thereto to precipitate crystals, and the result was filtered. Compound 1-b (26.8 g, 65.01 mmol, yield 80%) was obtained.

2) Preparation of Compound 1-c

Compound 1-b (20.0 g, 48.51 mmol, 1.0 eq.), 1-bromo-2-chlorobenzene (10.2 g, 53.36 mmol, 1.10 eq.), K₃PO₄ (20.6 g, 97.02 mmol, 2.0 eq.) and Pd(t-Bu₃P)₂ (0.12 g, 0.24 mmol, 0.005 eq.) were dissolved in toluene (160 mL), and stirred under reflux. When the reaction was finished, the solvent was removed under vacuum. After that, the result was completely dissolved in chloroform (CHCl₃), then washed with water, and the solvent was removed by about 50% under vacuum again. Ethanol was introduced thereto under reflux again to precipitate crystals, and the result was cooled and then filtered. Compound 1-c (20.0 g, 38.81 mmol, yield 80%) was obtained.

3) Preparation of Compound 1-d

Compound 1-c (15.0 g, 28.69 mmol, 1.0 eq.), Pd(dba)₂ (0.33 g, 0.57 mmol, 0.02 eq.), PCy₃ (0.35 g, 1.26 mmol, 0.04 eq.) and Cs₂CO₃ (20.6 g, 63.12 mmol, 2.0 eq.) were introduced to dimethylacetamide (95 mL), and stirred at 100° C. When the reaction was finished, the reaction material was poured into water to precipitate crystals, and the result was filtered. The filtered solid was completely dissolved in chloroform (CHCl₃), then washed with water, and the solvent was vacuum concentrated by about 80%. Methyl tert-butyl ether was introduced thereto to precipitate crystals, and the result was filtered. Compound 1-d (8.4 g, 17.21 mmol, yield 60%) was obtained.

4) Synthesis of Compound 1-e

Compound 1-d (8.0 g, 16.45 mmol, 1.0 eq.), bis(pinacolato)diboron (6.3 g, 24.67 mmol, 1.5 eq.), Pd(dba)₂ (0.19 g, 0.33 mmol, 0.02 eq.), PCy₃ (0.20 g, 0.72 mmol, 0.04 eq.) and KOAc (3.2 g, 32.9 mmol, 2.0 eq.) were dissolved in dioxane (55 mL), and stirred under reflux. When the reaction was finished, chloroform (CHCl₃) was introduced thereto, the result was filtered, and the solvent was removed under vacuum. After that, the result was completely dissolved in chloroform (CHCl₃), then washed with water, and the solvent was removed by about 50% under vacuum again. Ethanol was introduced thereto under reflux again to precipitate crystals, and the result was cooled and then filtered. Compound 1-e (7.5 g, 13.98 mmol, yield 85%) was obtained.

5) Synthesis of Compound 1-1

After dissolving Compound 1-e (7.0 g, 13.12 mmol, 1.0 eq.), 2-chloro-4,6-diphenyl-1,3,5-triazine (2.8 g, 14.43 mmol, 1.1 eq.) and Pd(PPh₃)₄ (0.15 g, 0.13 mmol, 0.01 eq.) in dioxane (30 mL) and stirring the mixture, K₃PO₄ (5.6 g, 26.25 mmol, 2.0 eq.) dissolved in water (10 mL) was added thereto, and the result was stirred under reflux. When the reaction was finished, EA (ethyl acetate) was introduced thereto, and after separating the organic layer, the solvent was removed under vacuum. The product was completely dissolved in chloroform (CHCl₃), then washed with water, and the solvent was removed by about 50% under vacuum. Ethyl acetate (EA) was introduced thereto under reflux again to precipitate crystals, and the result was cooled and then filtered. Compound 1-1 (7.5 g, 11.81 mmol, yield 90%) was obtained.

HR LC/MS/MS m/z calculated for C45H26N4O (M+): 638.2107; found: 638.2106

### [Preparation Example 2] Preparation of Compound 1-2

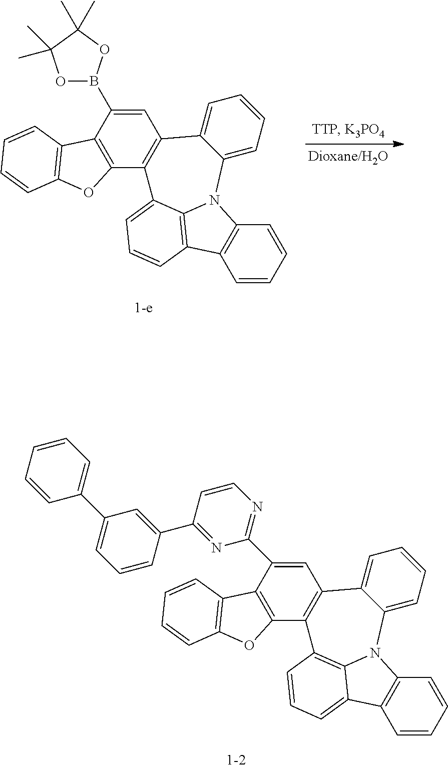 [SMILES: CC1(C)OB(c2cc3c(c4oc5ccccc5c24)-c2cccc4c5ccccc5n(c24)-c2ccccc2-3)OC1(C)C ; c1ccc(-c2cccc(-c3ccnc(-c4cc5c(c6oc7ccccc7c46)-c4cccc6c7ccccc7n(c46)-c4ccccc4-5)n3)c2)cc1]

After dissolving Compound 1-e (10.0 g, 18.75 mmol, 1.0 eq.), 4-([1,1′-biphenyl]-3-yl)-2-chloropyrimidine (5.5 g, 20.62 mmol, 1.1 eq.) and Pd(PPh₃)₄ (0.22 g, 0.19 mmol, 0.01 eq.) in dioxane (45 mL) and stirring the mixture, K₃PO₄ (8.0 g, 37.5 mmol, 2.0 eq.) dissolved in water (15 mL) was added thereto, and the result was stirred under reflux. When the reaction was finished, EA (ethyl acetate) was introduced thereto, and after separating the organic layer, the solvent was removed under vacuum. The product was completely dissolved in chloroform (CHCl₃), then washed with water, and the solvent was removed by about 50% under vacuum. Ethyl acetate (EA) was introduced thereto under reflux again to drop crystals, and the result was cooled and then filtered. Compound 1-2 (10.1 g, 15.93 mmol, yield 85%) was obtained.

HR LC/MS/MS m/z calculated for C46H27N3O (M+): 637.2154; found: 637.2155.

### [Preparation Example 3] Preparation of Compound 2-1

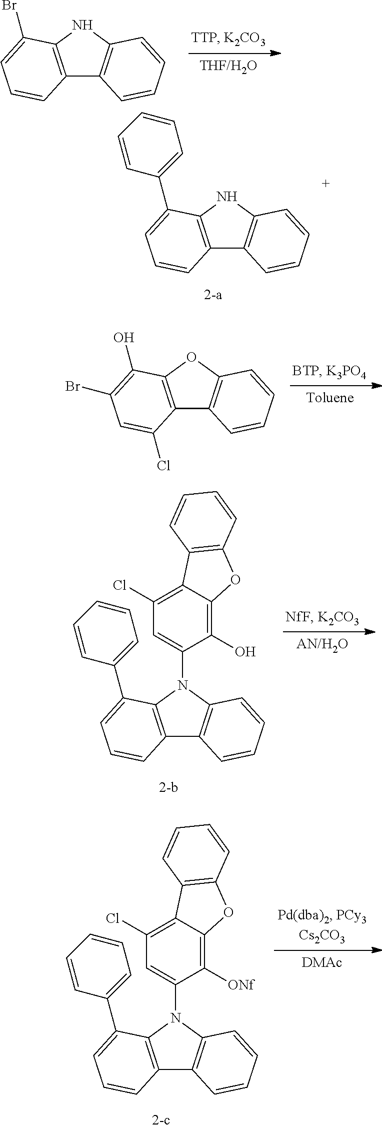

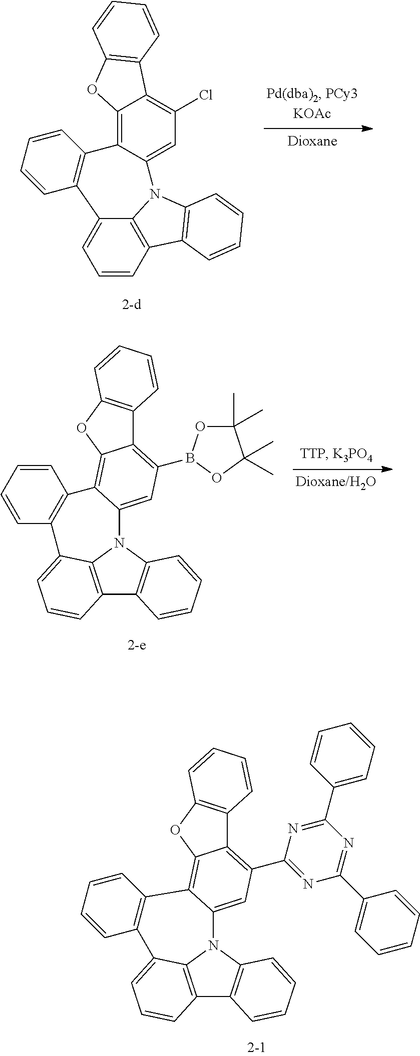 [SMILES: Clc1cc2c(c3oc4ccccc4c13)-c1ccccc1-c1cccc3c4ccccc4n-2c13 ; "Dioxane" ; CC1(C)OB(c2cc3c(c4oc5ccccc5c24)-c2ccccc2-c2cccc4c5ccccc5n-3c24)OC1(C)C ; "Dioxane/H2O" ; c1ccc(-c2nc(-c3ccccc3)nc(-c3cc4c(c5oc6ccccc6c35)-c3ccccc3-c3cccc5c6ccccc6n-4c35)n2)cc1]

1) Preparation of Compound 2-a

After dissolving 1-bromocarbazole (20.0 g, 81.26 mmol, 1.0 eq.), iodobenzene (17.4 g, 85.33 mmol, 1.05 eq.) and Pd(PPh₃)₄ (0.94 g, 0.81 mmol, 0.01 eq.) in tetrahydrofuran (THF) (200 mL) and stirring the mixture, K₂CO₃ (22.5 g, 162.53 mmol, 2.0 eq.) dissolved in water (50 mL) was added thereto, and the result was stirred under reflux. When the reaction was finished, EA (ethyl acetate) was introduced thereto, and after separating the organic layer, the solvent was removed under vacuum. The product was completely dissolved in chloroform (CHCl₃), then washed with water, and the solvent was vacuum concentrated by about 80%. Ethanol was introduced thereto to drop crystals, and the result was filtered. Compound 2-a (17.8 g, 73.14 mmol, yield 90%) was obtained.

2) Preparation of Compound 2-b

Compound 2-a (17.0 g, 69.87 mmol, 1.0 eq.), 3-bromo-1-chlorodibenzo[b,d]furan-4-ol (21.8 g, 73.36 mmol, 1.05 eq.), K₃PO₄ (29.7 g, 139.7 mmol, 2.0 eq.) and Pd(t-Bu₃P)₂ (0.18 g, 0.35 mmol, 0.005 eq.) were dissolved in toluene (230 mL), and stirred under reflux. When the reaction was finished, the solvent was removed under vacuum. After that, the result was completely dissolved in chloroform (CHCl₃), then washed with water, and the solvent was removed by about 50% under vacuum again. Ethanol was introduced thereto under reflux again to drop crystals, and the result was cooled and then filtered. Compound 2-b (24.1 g, 52.40 mmol, yield 75%) was obtained.

3) Preparation of Compound 2-c

Compound 2-b (20.0 g, 43.48 mmol, 1.0 eq.) and K₂CO₃ (12.0 g, 86.97 mmol, 2.0 eq.) were dissolved in acetonitrile (ACN) (100 mL) and H₂O (40 mL), and stirred. After that, NfF (nonafluorobutanesulfonyl fluoride) (11.7 mL, 65.23 mmol, 1.5 eq.) was slowly added thereto, and the result was stirred. When the reaction was finished, EA (ethyl acetate) was introduced thereto, and after separating the organic layer, the solvent was removed under vacuum. The product was completely dissolved in chloroform (CHCl₃), then washed with water, and the solvent was vacuum concentrated by about 80%. Methyl tert-butyl ether was introduced thereto to precipitate crystals, and the result was filtered. Compound 2-c (29.0 g, 39.14 mmol, yield 90%) was obtained.

4) Preparation of Compound 2-d

Compound 2-c (25.0 g, 33.69 mmol, 1.0 eq.), Pd(dba)₂ (0.39 g, 0.67 mmol, 0.02 eq.), PCy₃ (0.40 g, 0.40 mmol, 0.04 eq.) and Cs₂CO₃ (23.1 g, 70.8 mmol, 2.0 eq.) were introduced to dimethylacetamide (110 mL), and stirred at 100° C. When the reaction was finished, the reaction material was poured into water to precipitate crystals, and the result was filtered. The filtered solid was completely dissolved in chloroform (CHCl₃), then washed with water, and the solvent was vacuum concentrated by about 80%. Methyl tert-butyl ether was introduced thereto to precipitate crystals, and the result was filtered. Compound 2-d (9.7 g, 21.9 mmol, yield 65%) was obtained.

5) Synthesis of Compound 2-e

Compound 2-d (9.0 g, 20.37 mmol, 1.0 eq.), bis(pinacolato)diboron (7.7 g, 30.32 mmol, 1.5 eq.), Pd(dba)₂ (0.23 g, 0.41 mmol, 0.02 eq.), PCy₃ (0.24 g, 0.86 mmol, 0.04 eq.) and KOAc (4.0 g, 40.73 mmol, 2.0 eq.) were dissolved in dioxane (70 mL), and stirred under reflux. When the reaction was finished, chloroform (CHCl₃) was introduced thereto, the result was filtered, and the solvent was removed under vacuum. After that, the result was completely dissolved in chloroform (CHCl₃), then washed with water, and the solvent was removed by about 50% under vacuum again. Ethanol was introduced thereto under reflux again to precipitate crystals, and the result was cooled and then filtered. Compound 2-e (9.6 g, 17.92 mmol, yield 88%) was obtained.

6) Synthesis of Compound 2-1

After dissolving Compound 2-e (9.0 g, 16.9 mmol, 1.0 eq.), 2-chloro-4,6-diphenyl-1,3,5-triazine (5.0 g, 18.56 mmol, 1.1 eq.) and Pd(PPh₃)₄ (0.19 g, 0.17 mmol, 0.01 eq.) in dioxane (40 mL) and stirring the mixture, K₃PO₄ (7.2 g, 33.74 mmol, 2.0 eq.) dissolved in water (15 mL) was added thereto, and the result was stirred under reflux. When the reaction was finished, EA (ethyl acetate) was introduced thereto, and after separating the organic layer, the solvent was removed under vacuum. The product was completely dissolved in chloroform (CHCl₃), then washed with water, and the solvent was removed by about 50% under vacuum. Ethyl acetate (EA) was introduced thereto under reflux again to precipitate crystals, and the result was cooled and then filtered. Compound 2-1 (9.2 g, 14.34 mmol, yield 85%) was obtained.

HR LC/MS/MS m/z calculated for C45H26N4O (M+): 638.2107; found: 638.2109

### [Preparation Example 4] Preparation of Compound 2-2

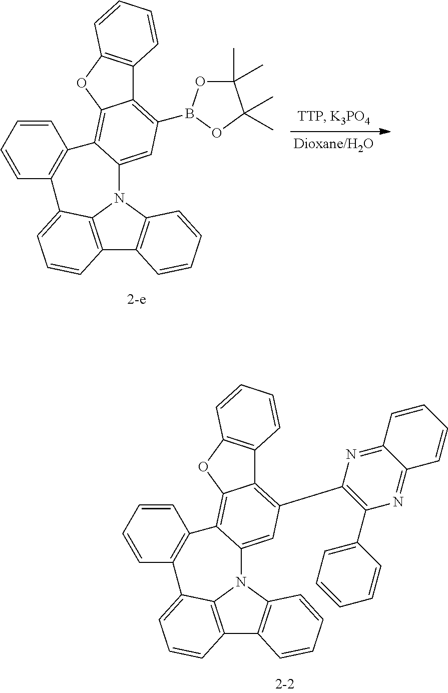 [SMILES: CC1(C)OB(c2cc3c(c4oc5ccccc5c24)-c2ccccc2-c2cccc4c5ccccc5n-3c24)OC1(C)C ; "Dioxane/H2O" ; c1ccc(-c2nc3ccccc3nc2-c2cc3c(c4oc5ccccc5c24)-c2ccccc2-c2cccc4c5ccccc5n-3c24)cc1]

After dissolving Compound 2-e (10.0 g, 18.75 mmol, 1.0 eq.), 2-chloro-3-phenylquinoxaline (5.0 g, 20.62 mmol, 1.1 eq.) and Pd(PPh₃)₄ (0.22 g, 0.19 mmol, 0.01 eq.) in dioxane (45 mL) and stirring the mixture, K₃PO₄ (8.0 g, 37.5 mmol, 2.0 eq.) dissolved in water (15 mL) was added thereto, and the result was stirred under reflux. When the reaction was finished, EA (ethyl acetate) was introduced thereto, and after separating the organic layer, the solvent was removed under vacuum. The product was completely dissolved in chloroform (CHCl₃), then washed with water, and the solvent was removed by about 50% under vacuum. Ethyl acetate (EA) was introduced thereto under reflux again to precipitate crystals, and the result was cooled and then filtered. Compound 2-2 (9.2 g, 15.0 mmol, yield 80%) was obtained.

HR LC/MS/MS m/z calculated for C44H25N3O (M+): 611.1998; found: 611.1998

### [Preparation Example 5] Preparation of Compound 2-3

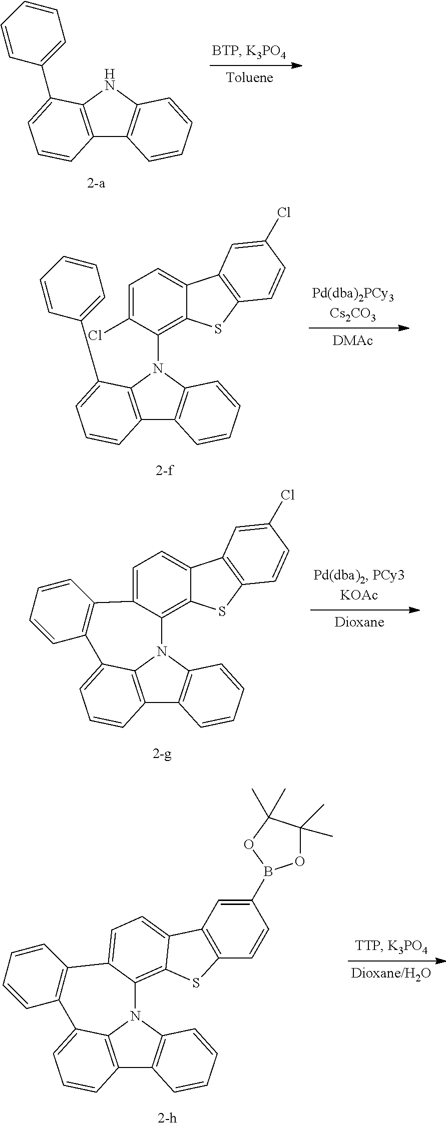

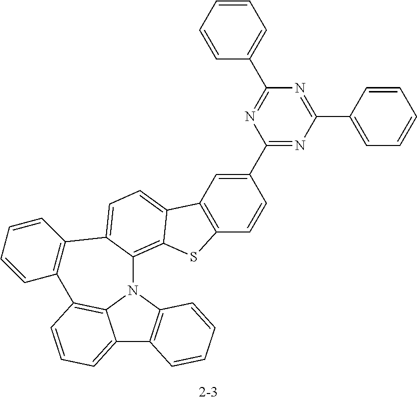 [SMILES: c1ccc(-c2nc(-c3ccccc3)nc(-c3ccc4sc5c6c(ccc5c4c3)-c3ccccc3-c3cccc4c5ccccc5n-6c34)n2)cc1]

1) Preparation of Compound 2-f

Compound 2-a (15.0 g, 61.65 mmol, 1.0 eq.), 3,8-dichlorodibenzo[b,d]thiophen-4-yl-1,1,2,2,3,3,4,4,4-nonafluorobutane-1-sulfonate (37.4 g, 67.8 mmol, 1.1 eq.), K₃PO₄ (26.2 g, 123.3 mmol, 2.0 eq.) and Pd(t-Bu₃P)₂ (0.16 g, 0.31 mmol, 0.005 eq.) were dissolved in toluene (200 mL), and stirred under reflux. When the reaction was finished, the solvent was removed under vacuum. After that, the result was completely dissolved in chloroform (CHCl₃), then washed with water, and the solvent was removed by about 50% under vacuum again. Methanol was introduced thereto under reflux again to precipitate crystals, and the result was cooled and then filtered. Compound 2-f (24.4 g, 49.32 mmol, yield 80%) was obtained.

2) Preparation of Compound 2-g

Compound 2-f (20.0 g, 42.6 mmol, 1.0 eq.), Pd(dba)₂ (0.49 g, 0.85 mmol, 0.02 eq.), PCy₃ (0.53 g, 1.87 mmol, 0.04 eq.) and Cs₂CO₃ (30.5 g, 93.7 mmol, 2.0 eq.) was introduced to dimethylacetamide (140 mL), and stirred at 100° C. When the reaction was finished, the reaction material was poured into water to precipitate crystals, and the result was filtered. The filtered solid was completely dissolved in chloroform (CHCl₃), then washed with water, and the solvent was vacuum concentrated by about 80%. n-Hexane was introduced thereto to precipitate crystals, and the result was filtered. Compound 2-g (12.7 g, 27.7 mmol, yield 65%) was obtained.

3) Synthesis of Compound 2-h

Compound 2-g (12.0 g, 26.2 mmol, 1.0 eq.), bis(pinacolato)diboron (10.0 g, 17.03 mmol, 1.5 eq.), Pd(dba)₂ (0.30 g, 0.26 mmol, 0.02 eq.), PCy₃ (0.32 g, 1.15 mmol, 0.04 eq.) and KOAc (5.14 g, 52.4 mmol, 2.0 eq.) were dissolved in dioxane (85 mL), and stirred under reflux. When the reaction was finished, chloroform (CHCl₃) was introduced thereto, the result was filtered, and the solvent was removed under vacuum. After that, the result was completely dissolved in chloroform (CHCl₃), then washed with water, and the solvent was removed by about 50% under vacuum again. Ethanol was introduced thereto under reflux again to precipitate crystals, and the result was cooled and then filtered. Compound 2-h (12.2 g, 22.3 mmol, yield 85%) was obtained.

4) Synthesis of Compound 2-3

After dissolving Compound 2-h (12.0 g, 21.8 mmol, 1.0 eq.), 2-chloro-4,6-diphenyl-1,3,5-triazine (6.4 g, 24.0 mmol, 1.1 eq.) and Pd(PPh₃)₄ (0.25 g, 0.22 mmol, 0.01 eq.) in dioxane (55 mL) and stirring the mixture, K₃PO₄ (9.3 g, 43.7 mmol, 2.0 eq.) dissolved in water (15 mL) was added thereto, and the result was stirred under reflux. When the reaction was finished, EA (ethyl acetate) was introduced thereto, and after separating the organic layer, the solvent was removed under vacuum. The product was completely dissolved in chloroform (CHCl₃), then washed with water, and the solvent was removed by about 50% under vacuum. Ethyl acetate (EA) was introduced thereto under reflux again to precipitate crystals, and the result was cooled and then filtered. Compound 2-3 (11.0 g, 17.5 mmol, yield 80%) was obtained.

HR LC/MS/MS m/z calculated for C45H26N4S (M+): 654.1878; found: 654.1880

### [Preparation Example 6] Preparation of Compound 3-1

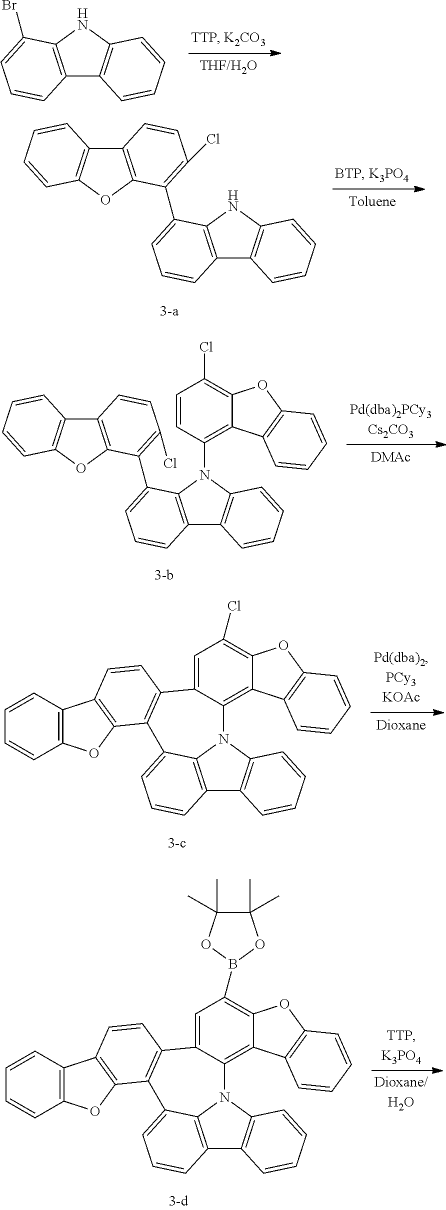

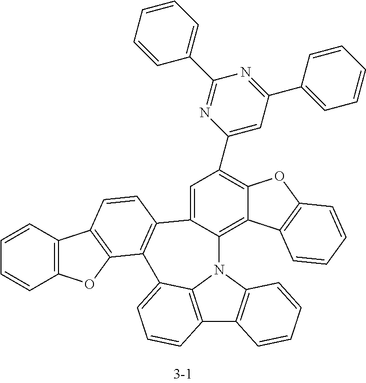 [SMILES: c1ccc(-c2cc(-c3cc4c(c5c3oc3ccccc35)-n3c5ccccc5c5cccc(c53)-c3c-4ccc4c3oc3ccccc34)nc(-c3ccccc3)n2)cc1]

1) Preparation of Compound 3-a

After dissolving 1-bromocarbazole (20.0 g, 81.26 mmol, 1.0 eq.), 3-chlorodibenzo[b,d]furan-4-yl 1,1,2,2,3,3,4,4,4-nonafluorobutanel-sulfonate (42.7 g, 85.33 mmol, 1.05 eq.) and Pd(PPh₃)₄ (0.94 g, 0.81 mmol, 0.01 eq.) in tetrahydrofuran (THF) (200 mL) and stirring the mixture, K₂CO₃ (22.5 g, 162.53 mmol, 2.0 eq.) dissolved in water (50 mL) was added thereto, and the result was stirred under reflux. When the reaction was finished, EA (ethyl acetate) was introduced thereto, and after separating the organic layer, the solvent was removed under vacuum. The product was completely dissolved in chloroform (CHCl₃), then washed with water, and the solvent was vacuum concentrated by about 80%. Ethanol was introduced thereto to precipitate crystals, and the result was filtered. Compound 3-a (20.9 g, 56.89 mmol, yield 70%) was obtained.

2) Preparation of Compound 3-b

Compound 3-a (20.0 g, 54.37 mmol, 1.0 eq.), 4-chlorodibenzo[b,d]furan-1-yl 1,1,2,2,3,3,4,4,4-nonafluoro-butane-1-sulfonate (30.0 g, 59.81 mmol, 1.1 eq.), K₃PO₄ (23.1 g, 108.8 mmol, 2.0 eq.) and Pd(t-Bu₃P)₂ (0.14 g, 0.27 mmol, 0.005 eq.) were dissolved in toluene (180 mL), and stirred under reflux. When the reaction was finished, the solvent was removed under vacuum. After that, the result was completely dissolved in chloroform (CHCl₃), then washed with water, and the solvent was removed by about 50% under vacuum again. Ethanol was introduced thereto under reflux again to precipitate crystals, and the result was cooled and then filtered. Compound 3-b (33.6 g, 46.2 mmol, yield 85%) was obtained.

3) Preparation of Compound 3-c

Compound 3-b (30.0 g, 41.2 mmol, 1.0 eq.), Pd(dba)₂ (0.47 g, 0.82 mmol, 0.02 eq.), PCy₃ (0.51 g, 1.81 mmol, 0.04 eq.) and Cs₂CO₃ (29.6 g, 90.7 mmol, 2.0 eq.) were introduced to dimethylacetamide (140 mL), and stirred at 100° C. When the reaction was finished, the reaction material was poured into water to precipitate crystals, and the result was filtered. The filtered solid was completely dissolved in chloroform (CHCl₃), then washed with water, and the solvent was vacuum concentrated by about 80%. Acetonitrile was introduced thereto to precipitate crystals, and the result was filtered. Compound 3-c (15.4 g, 28.9 mmol, yield 70%) was obtained.

4) Preparation of Compound 3-d

Compound 3-c (15.0 g, 28.2 mmol, 1.0 eq.), bis(pinacolato)diboron (10.7 g, 42.3 mmol, 1.5 eq.), Pd(dba)₂ (0.32 g, 0.56 mmol, 0.02 eq.), PCy₃ (0.35 g, 1.24 mmol, 0.04 eq.) and KOAc (5.53 g, 56.4 mmol, 2.0 eq.) were dissolved in dioxane (90 mL), and stirred under reflux. When the reaction was finished, chloroform (CHCl₃) was introduced thereto, the result was filtered, and the solvent was removed under vacuum. After that, the result was completely dissolved in chloroform (CHCl₃), then washed with water, and the solvent was removed by about 50% under vacuum again. Ethanol was introduced thereto under reflux again to precipitate crystals, and the result was cooled and then filtered. Compound 3-d (14.8 g, 23.7 mmol, yield 84%) was obtained.

5) Preparation of Compound 3-1

After dissolving Compound 3-d (20.0 g, 32.08 mmol, 1.0 eq.), 4-chloro-2,6-diphenylpyrimidine (9.5 g, 35.3 mmol, 1.1 eq.) and Pd(PPh₃)₄ (0.37 g, 0.64 mmol, 0.01 eq.) in dioxane (70 mL) and stirring the mixture, K₃PO₄ (13.6 g, 64.2 mmol, 2.0 eq.) dissolved in water (30 mL) was added thereto, and the result was stirred under reflux. When the reaction was finished, EA (ethyl acetate) was introduced thereto, and after separating the organic layer, the solvent was removed under vacuum. The product was completely dissolved in chloroform (CHCl₃), then washed with water, and the solvent was removed by about 50% under vacuum. Ethyl acetate (EA) was introduced thereto under reflux again to precipitate crystals, and the result was cooled and then filtered. Compound 3-1 (19.8 g, 27.3 mmol, yield 85%) was obtained.

HR LC/MS/MS m/z calculated for C46H25N3O₂ (M+): 651.1947; found: 651.1948.

### [Preparation Example 7] Preparation of Compound 3-2

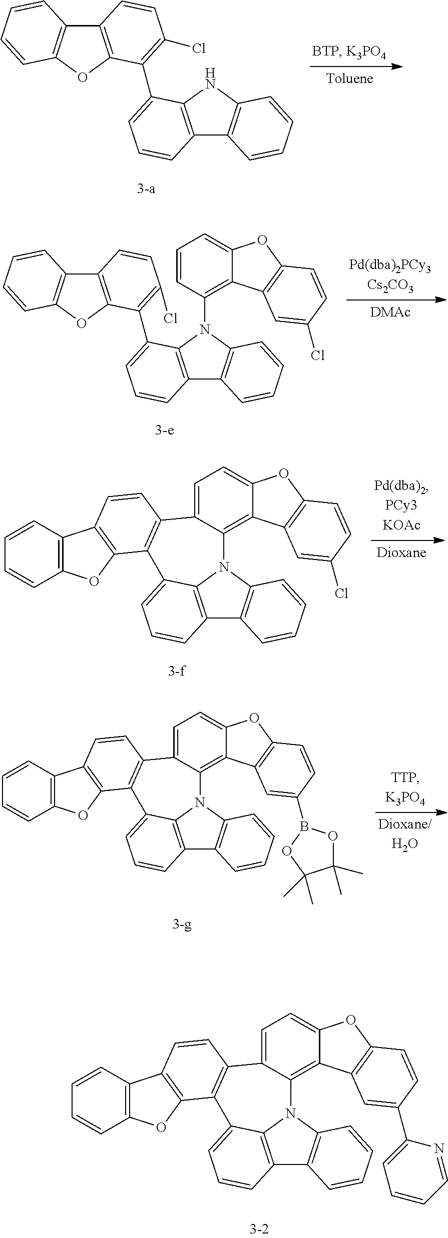

1) Preparation of Compound 3-e

Compound 3-a (20.0 g, 54.4 mmol, 1.0 eq.), 1-bromo-8-chlorodibenzo[b,d]furan (16.8 g, 59.8 mmol, 1.1 eq.), K₃PO₄ (23.1 g, 108.8 mmol, 2.0 eq.) and Pd(t-Bu₃P)₂ (0.14 g, 0.27 mmol, 0.005 eq.) were dissolved in toluene (180 mL), and stirred under reflux. When the reaction was finished, the solvent was removed under vacuum. After that, the result was completely dissolved in chloroform (CHCl₃), then washed with water, and the solvent was removed by about 50% under vacuum again. Methanol was introduced thereto under reflux again to precipitate crystals, and the result was cooled and then filtered. Compound 3-e (21.6 g, 38.1 mmol, yield 70%) was obtained.

2) Preparation of Compound 3-f

Compound 3-e (20.0 g, 35.2 mmol, 1.0 eq.), Pd(dba)₂ (0.4 g, 0.70 mmol, 0.02 eq.), PCy₃ (0.43 g, 1.55 mmol, 0.04 eq.) and Cs₂CO₃ (25.2 g, 77.4 mmol, 2.0 eq.) were introduced to dimethylacetamide (110 mL), and stirred at 100° C. When the reaction was finished, the reaction material was poured into water to precipitate crystals, and the result was filtered. The filtered solid was completely dissolved in chloroform (CHCl₃), then washed with water, and the solvent was vacuum concentrated by about 50%. Ethanol was introduced thereto to precipitate crystals, and the result was filtered. Compound 3-f (13.1 g, 24.6 mmol, yield 70%) was obtained.

3) Preparation of Compound 3-g

Compound 3-f (13.0 g, 24.4 mmol, 1.0 eq.), bis(pinacolato)diboron (9.3 g, 36.6 mmol, 1.5 eq.), Pd(dba)₂ (0.28 g, 0.49 mmol, 0.02 eq.), PCy₃ (0.30 g, 1.08 mmol, 0.04 eq.) and KOAc (4.80 g, 48.9 mmol, 2.0 eq.) were dissolved in dioxane (80 mL), and stirred under reflux. When the reaction was finished, chloroform (CHCl₃) was introduced thereto, the result was filtered, and the solvent was removed under vacuum. After that, the result was completely dissolved in chloroform (CHCl₃), then washed with water, and the solvent was removed by about 50% under vacuum again. Methyl tert-butyl ether was introduced thereto under reflux again to precipitate crystals, and the result was cooled and then filtered. Compound 3-g (10.7 g, 17.1 mmol, yield 80%) was obtained.

4) Preparation of Compound 3-2

After dissolving Compound 3-g (10.0 g, 16.0 mmol, 1.0 eq.), 2-bromopyridine (2.7 g, 16.8 mmol, 1.05 eq.) and Pd(PPh₃)₄ (0.19 g, 0.16 mmol, 0.01 eq.) in dioxane (40 mL) and stirring the mixture, K₃PO₄ (6.81 g, 32.1 mmol, 2.0 eq.) dissolved in water (10 mL) was added thereto, and the result was stirred under reflux. When the reaction was finished, EA (ethyl acetate) was introduced thereto, and after separating the organic layer, the solvent was removed under vacuum. The product was completely dissolved in chloroform (CHCl₃), then washed with water, and the solvent was removed by about 50% under vacuum. Ethyl acetate (EA) was introduced thereto under reflux again to precipitate crystals, and the result was cooled and then filtered. Compound 3-2 (8.3 g, 14.4 mmol, yield 90%) was obtained.

HR LC/MS/MS m/z calculated for C41H22N2O₂ (M+): 574.1681; found: 574.1681

### [Preparation Example 8] Preparation of Compound 3-3

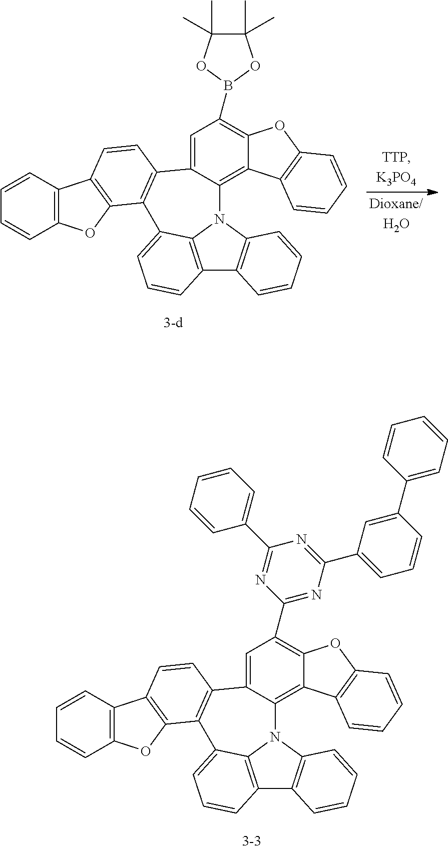 [SMILES: CC1(C)OB(c2cc3c(c4c2oc2ccccc24)-n2c4ccccc4c4cccc(c42)-c2c-3ccc3c2oc2ccccc23)OC1(C)C ; "Dioxane/H2O" ; c1ccc(-c2cccc(-c3nc(-c4ccccc4)nc(-c4cc5c(c6c4oc4ccccc46)-n4c6ccccc6c6cccc(c64)-c4c-5ccc5c4oc4ccccc45)n3)c2)cc1]

After dissolving Compound 3-d (10.0 g, 16.04 mmol, 1.0 eq.), 2-([1,1′-biphenyl]-3-yl)-4-chloro-6-phenyl-1,3,5-triazine (5.80 g, 16.8 mmol, 1.05 eq.) and Pd(PPh₃)₄ (0.19 g, 0.16 mmol, 0.01 eq.) in dioxane (40 mL) and stirring the mixture, K₃PO₄ (6.81 g, 32.1 mmol, 2.0 eq.) dissolved in water (10 mL) was added thereto, and the result was stirred under reflux. When the reaction was finished, EA (ethyl acetate) was introduced thereto, and after separating the organic layer, the solvent was removed under vacuum. The product was completely dissolved in chloroform (CHCl₃), then washed with water, and the solvent was removed by about 50% under vacuum. Ethyl acetate (EA) was introduced thereto under reflux again to precipitate crystals, and the result was cooled and then filtered. Compound 3-3 (11.4 g, 14.1 mmol, yield 88%) was obtained.

HR LC/MS/MS m/z calculated for C57H32N4O2 (M+): 804.2525; found: 804.2528

### [Preparation Example 9] Preparation of Compound 4-1

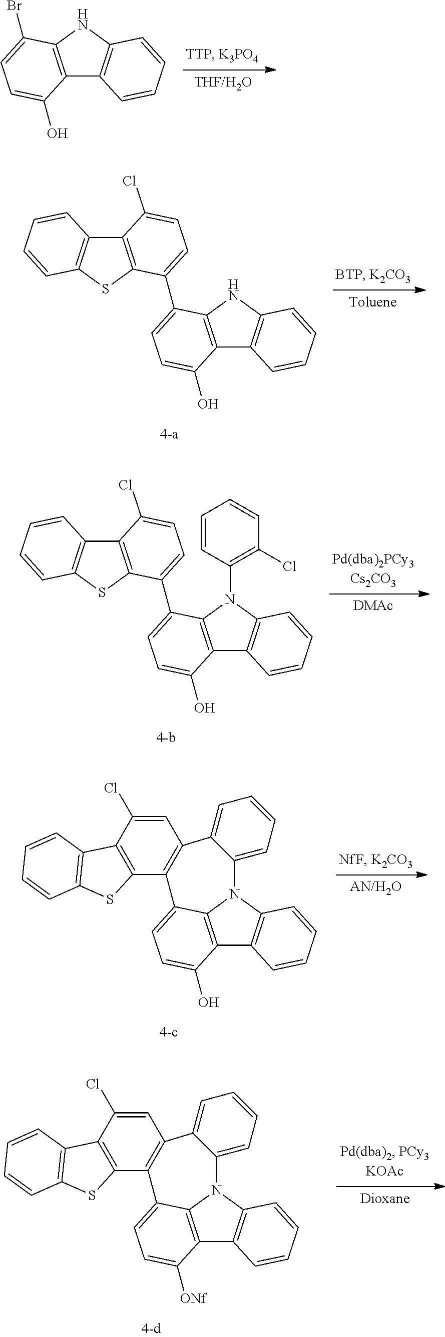

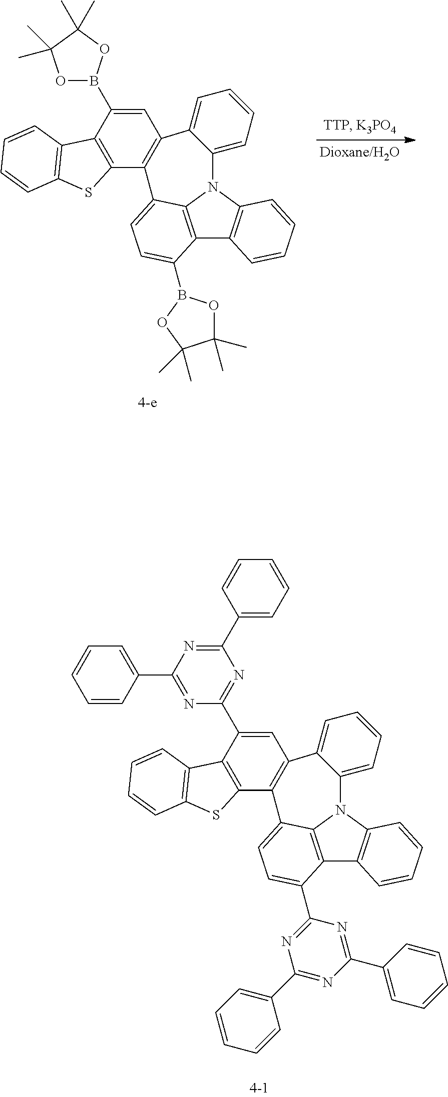 [SMILES: CC1(C)OB(c2cc3c(c4sc5ccccc5c24)-c2ccc(B4OC(C)(C)C(C)(C)O4)c4c5ccccc5n(c24)-c2ccccc2-3)OC1(C)C ; "Dioxane/H2O" ; c1ccc(-c2nc(-c3ccccc3)nc(-c3cc4c(c5sc6ccccc6c35)-c3ccc(-c5nc(-c6ccccc6)nc(-c6ccccc6)n5)c5c6ccccc6n(c35)-c3ccccc3-4)n2)cc1]

1) Preparation of Compound 4-a

After dissolving 1-bromo-9H-carbazol-4-ol (20.0 g, 76.30 mmol, 1.0 eq.), 1-chlorodibenzo[b,d]thiophen-4-yl-1,1,2,2,3,3,4,4,4-nonafluorobutanel-sulfonate (41.4 g, 80.1 mmol, 1.05 eq.) and Pd(PPh₃)₄ (0.88 g, 0.76 mmol, 0.01 eq.) in tetrahydrofuran (THF) (200 mL) and stirring the mixture, K₂CO₃ (21.1 g, 152.6 mmol, 2.0 eq.) dissolved in water (50 mL) was added thereto, and the result was stirred under reflux. When the reaction was finished, EA (ethyl acetate) was introduced thereto, and after separating the organic layer, the solvent was removed under vacuum. The product was completely dissolved in chloroform (CHCl₃), then washed with water, and the solvent was vacuum concentrated by about 80%. Ethanol was introduced thereto to precipitate crystals, and the result was filtered. Compound 4-a (22.9 g, 57.2 mmol, yield 75%) was obtained.

2) Preparation of Compound 4-b

Compound 4-a (20.0 g, 50.01 mmol, 1.0 eq.), 1-bromo-2-chlorobenzene (10.1 g, 52.5 mmol, 1.1 eq.), K₃PO₄ (21.2 g, 100.0 mmol, 2.0 eq.) and Pd(t-Bu₃P)₂ (0.13 g, 0.25 mmol, 0.005 eq.) were dissolved in toluene (160 mL), and stirred under reflux. When the reaction was finished, the solvent was removed under vacuum. After that, the result was completely dissolved in chloroform (CHCl₃), then washed with water, and the solvent was removed by about 50% under vacuum again. Ethanol was introduced thereto under reflux again to precipitate crystals, and the result was cooled and then filtered. Compound 4-b (20.4 g, 40.0 mmol, yield 80%) was obtained.

3) Preparation of Compound 4-c

Compound 4-b (20.0 g, 39.2 mmol, 1.0 eq.), Pd(dba)₂ (0.45 g, 0.78 mmol, 0.02 eq.), PCy₃ (0.46 g, 1.65 mmol, 0.04 eq.) and Cs₂CO₃ (26.8 g, 82.3 mmol, 2.0 eq.) was introduced to dimethylacetamide (130 mL), and stirred at 100° C. When the reaction was finished, the reaction material was poured into water to precipitate crystals, and the result was filtered. The filtered solid was completely dissolved in chloroform (CHCl₃), then washed with water, and the solvent was vacuum concentrated by about 80%. Ethanol was introduced thereto to precipitate crystals, and the result was filtered. Compound 4-c (14.5 g, 30.6 mmol, yield 78%) was obtained.

4) Preparation of Compound 4-d

Compound 4-c (13.0 g, 27.4 mmol, 1.0 eq.) and K₂CO₃ (11.4 g, 82.3 mmol, 3.0 eq.) were dissolved in acetonitrile (AN) (70 mL) and H₂O (20 mL), and stirred. After that, NfF (nonafluorobutanesulfonyl fluoride) (7.40 mL, 41.1 mmol, 1.5 eq.) was slowly added thereto, and the result was stirred. When the reaction was finished, EA (ethyl acetate) was introduced thereto, and after separating the organic layer, the solvent was removed under vacuum. The product was completely dissolved in chloroform (CHCl₃), then washed with water, and the solvent was vacuum concentrated by about 80%. Methyl tert-butyl ether was introduced thereto to precipitate crystals, and the result was filtered. Compound 4-d (18.8 g, 24.7 mmol, yield 90%) was obtained.

5) Preparation of Compound 4-e

Compound 4-d (15.0 g, 19.7 mmol, 1.0 eq.), bis(pinacolato)diboron (15.0 g, 59.2 mmol, 3.0 eq.), Pd(dba)₂ (0.45 g, 0.79 mmol, 0.04 eq.), PCy₃ (0.46 g, 1.66 mmol, 0.08 eq.) and KOAc (5.81 g, 59.2 mmol, 3.0 eq.) were dissolved in dioxane (70 mL), and stirred under reflux. When the reaction was finished, chloroform (CHCl₃) was introduced thereto, the result was filtered, and the solvent was removed under vacuum. After that, the result was completely dissolved in chloroform (CHCl₃), then washed with water, and the solvent was removed by about 50% under vacuum again. Ethanol was introduced thereto under reflux again to precipitate crystals, and the result was cooled and then filtered. Compound 4-e (10.2 g, 15.4 mmol, yield 78%) was obtained.

6) Preparation of Compound 4-1

After dissolving Compound 4-e (10.0 g, 15.2 mmol, 1.0 eq.), 2-chloro-4,6-diphenyl-1,3,5-triazine (8.93 g, 33.4 mmol, 2.2 eq.) and Pd(PPh₃)₄ (0.35 g, 0.30 mmol, 0.02 eq.) in dioxane (50 mL) and stirring the mixture, K₃PO₄ (9.70 g, 45.5 mmol, 3.0 eq.) dissolved in water (15 mL) was added thereto, and the result was stirred under reflux. When the reaction was finished, EA (ethyl acetate) was introduced thereto, and after separating the organic layer, the solvent was removed under vacuum. The product was completely dissolved in chloroform (CHCl₃), then washed with water, and the solvent was removed by about 50% under vacuum. Ethyl acetate (EA) was introduced thereto under reflux again to precipitate crystals, and the result was cooled and then filtered. Compound 4-1 (9.20 g, 10.6 mmol, yield 70%) was obtained.

HR LC/MS/MS m/z calculated for C60H35N7S (M+): 885.2675; found: 885.2675

### EXAMPLES

### Example 1

A glass substrate on which ITO (indium tin oxide) was coated as a thin film to a thickness of 1,300 Å was placed in distilled water containing dissolved detergent and ultrasonically cleaned. Herein, a product of Fischer Co. was used as the detergent, and as the distilled water, distilled water filtered twice with a filter manufactured by Millipore Co. was used. After the ITO was cleaned for 30 minutes, ultrasonic cleaning was repeated twice using distilled water for 10 minutes. After the cleaning with distilled water was finished, the substrate was ultrasonically cleaned with solvents of isopropyl alcohol, acetone and methanol, then dried, and then transferred to a plasma cleaner. The substrate was cleaned for 5 minutes using oxygen plasma, and then transferred to a vacuum deposition apparatus.

On the transparent ITO electrode prepared as above, the following Compound HI-1 was thermal vacuum deposited to a thickness of 50 Å to form a hole injection layer.

A hole transfer layer was formed on the hole injection layer by thermal vacuum depositing the following Compound HT-1 to a thickness of 250 Å, and on the HT-1 deposition film, the following Compound HT-2 was vacuum deposited to a thickness of 50 Å to form an electron blocking layer.

Subsequently, Compound 1-1 prepared in Synthesis Example 1 was deposited on the HT-2 deposition film to a thickness of 400 Å, and the following Phosphorescent Dopant GD-1 was co-deposited in a weight ratio of 5% to 20% to form a light emitting layer.

On the light emitting layer, the following ET-1 material was vacuum deposited to a thickness of 250 Å, and in addition thereto, the following ET-2 material was co-deposited with a 2% weight ratio of L1 to a thickness of 100 Å to form an electron transfer layer and an electron injection layer. A cathode was formed on the electron injection layer by depositing aluminum to a thickness of 1000 Å.

In the above-described process, the deposition rates of the organic materials were maintained at 0.4 Å/sec to 0.7 Å/sec, the deposition rate of the aluminum was maintained at 2 Å/sec, and the degree of vacuum during the deposition was maintained at 5×10⁻⁸ torr to 1×10⁻⁷ torr.

### Example 2 to Example 5

Organic light emitting devices of Examples 2 to 5 were each manufactured in the same manner as in Example 1 except that, when forming the light emitting layer, the content of the phosphorescent host material and the content of the dopant were changed as in the following Table 1.

### Comparative Example 1 to Comparative Example 3

Organic light emitting devices of Comparative Examples 1 to 3 were each manufactured in the same manner as in Example 1 except that, when forming the light emitting layer, the content of the phosphorescent host material and the content of the dopant were changed as in the following Table 1. Herein, the host materials used in Comparative Examples 1 to 3 are Compounds A to C as follows.

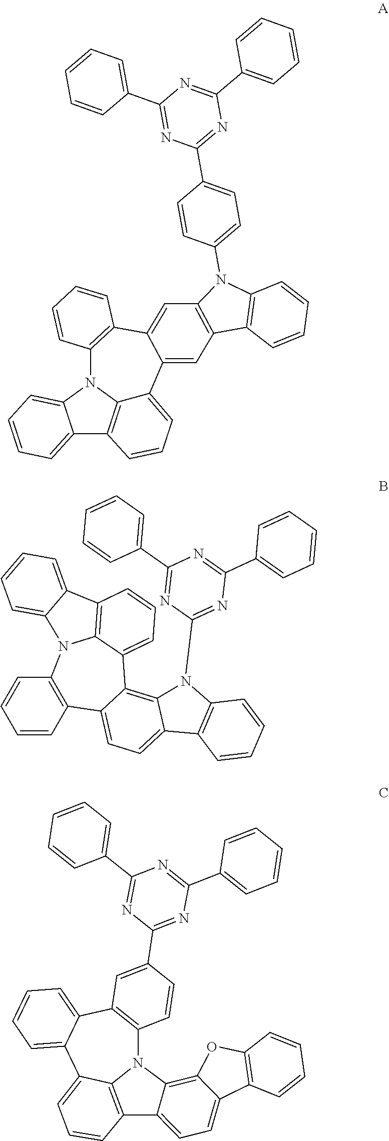

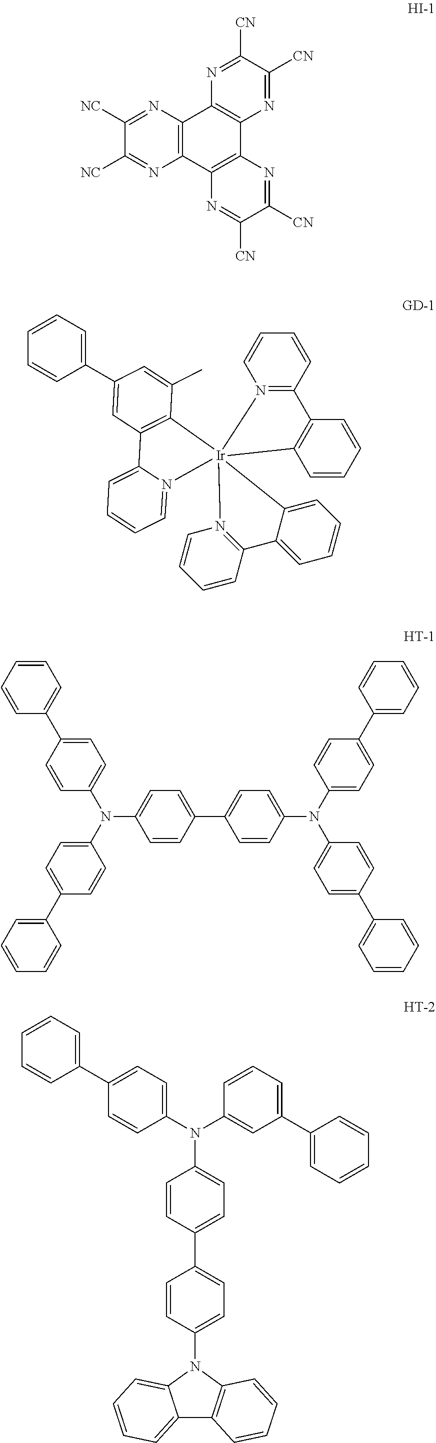

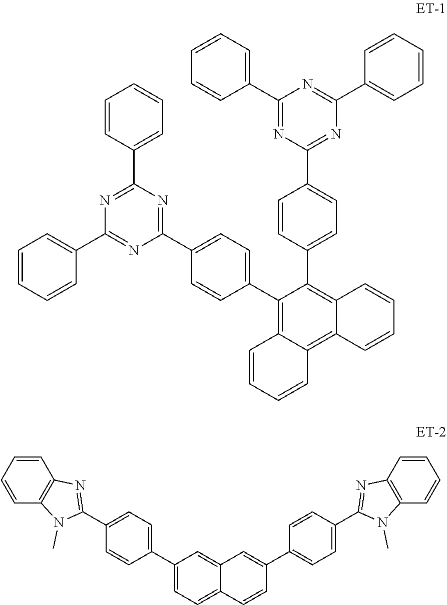 [SMILES: c1ccc(-c2nc(-c3ccccc3)nc(-c3ccc(-c4c(-c5ccc(-c6nc(-c7ccccc7)nc(-c7ccccc7)n6)cc5)c5ccccc5c5ccccc45)cc3)n2)cc1 ; Cn1c(-c2ccc(-c3ccc4ccc(-c5ccc(-c6nc7ccccc7n6C)cc5)cc4c3)cc2)nc2ccccc21]

### EXPERIMENTAL EXAMPLE

By applying a current to each of the organic light emitting devices manufactured in Examples 1 to 5 and Comparative Examples 1 to 3, voltage, efficiency, color coordinate and lifetime were measured, and the results are shown in the following Table 1. Herein, T₉₅ means a time taken for luminance to decrease to 95% when initial luminance at optical density of 20 mA/cm² is employed as 100%.

TABLE 1

| | | Voltage | | | Lifetime |
|---|---|---|---|---|---|
| | Host:Dopant | (V) | EQE (%) | Color | (T₉₅, h) |
| | (Thickness, Å) | (@10 | (@10 | Coordinate | (@20 |
| Entry | Dopant Content | mA/ cm²) | mA/ cm²) | (x, y) | mA/cm²) |
| Example 1 | Compound 1-1:GD-1 | 3.20 | 18.0 | (0.32, 0.62) | 75.1 |
| | (400) 12% | | | | |
| Example 2 | Compound 1-2:GD-1 | 3.13 | 18.7 | (0.32, 0.61) | 74.3 |
| | (400) 12% | | | | |
| Example 3 | Compound 2-1:GD-1 | 3.27 | 17.9 | (0.34, 0.62) | 72.1 |
| | (400) 12% | | | | |
| Example 4 | Compound 2-3:GD-1 | 3.28 | 19.7 | (0.32, 0.63) | 70.4 |
| | (400) 12% | | | | |
| Example 5 | Compound 4-1:GD-1 | 3.28 | 19.7 | (0.32, 0.64) | 90.4 |
| | (400) 12% | | | | |
| Comparative | Compound A: GD-1 | 4.70 | 13.2 | (0.33, 0.61) | 60.9 |
| Example 1 | (400) 12% | | | | |
| Comparative | Compound B:GD-1 | 3.43 | 15.5 | (0.34, 0.62) | 57.6 |
| Example 2 | (400) 12% | | | | |
| Comparative | Compound C:GD-1 | 3.52 | 16.7 | (0.34, 0.62) | 65.2 |
| Example 3 | (400) 12% | | | | |

Comparative Examples 1 and 2 are compounds in which one of X1 and X2 of Chemical Formula 2 bonding to Chemical Formula 1 of the present disclosure is N, and the other one is a direct bond. Comparative Example 3 is a compound in which Chemical Formula 2 substitutes at a part other than the bonding part indicated in Chemical Formula 1 of the present disclosure. As shown in the table, it was seen that Examples 1 to 5 using the heterocyclic compound of the present disclosure as a host had effects of low voltage, high efficiency and long lifetime compared to Comparative Examples 1 to 3.

1. A heterocyclic compound of Chemical Formula 1: 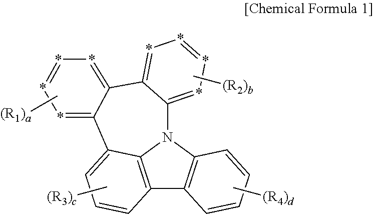
wherein, in Chemical Formula 1:
adjacent one or more pairs of * bond to X₁ and X₂ of the following Chemical Formula 2: 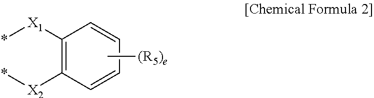
wherein in Chemical Formula 2:
* means the part of Chemical Formula 1 bonding to X₁ and X₂;
any one of X₁ and X₂ is a direct bond;
when X₁ is a direct bond, X₂ is CRaRb, O or S;
when X₂ is a direct bond, X₁ is CRaRb, O or S; and
at least one of R₁ to R₅ is the following Chemical Formula 3, and the rest that are not Chemical Formula 3 are the same as or different from each other and each independently is hydrogen, deuterium, a substituted or unsubstituted alkyl group, a substituted or unsubstituted aryl group, a substituted or unsubstituted heteroaryl group, a substituted or unsubstituted cycloalkyl group, a substituted or unsubstituted alkoxy group or a combination thereof; 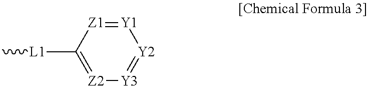
wherein in Chemical Formula 3: [symbol]
is a part bonding to the core of Chemical Formula 1;
at least one of Z1 and Z2 is N, and the other one is CRc;
Y1 to Y3 are the same as or different from each other, and each independently is CRd or N;
L1 is a direct bond, a substituted or unsubstituted alkylene group, a substituted or unsubstituted arylene group, a substituted or unsubstituted heteroarylene group or a combination thereof,
Ra to Rd are the same as or different from each other, and each independently is hydrogen, deuterium, a substituted or unsubstituted alkyl group, a substituted or unsubstituted aryl group, a substituted or unsubstituted heteroaryl group, a substituted or unsubstituted cycloalkyl group, a substituted or unsubstituted alkoxy group or a combination thereof,
Rd bonds to adjacent substituents to form a substituted or unsubstituted ring;
a is an integer of 0 to 4, and when a is 2 or greater, the R₁s are the same as or different from each other;
b is an integer of 0 to 4, and when b is 2 or greater, the R₂s are the same as or different from each other;
c is an integer of 0 to 3, and when c is 2 or greater, the R₃s are the same as or different from each other;
d is an integer of 0 to 4, and when d is 2 or greater, the R₄s are the same as or different from each other; and
e is an integer of 0 to 4, and when e is 2 or greater, the R₅s are the same as or different from each other.

2. The heterocyclic compound of claim 1, wherein Chemical Formula 1 is the following Chemical Formula 1-1 or Chemical Formula 1-2: 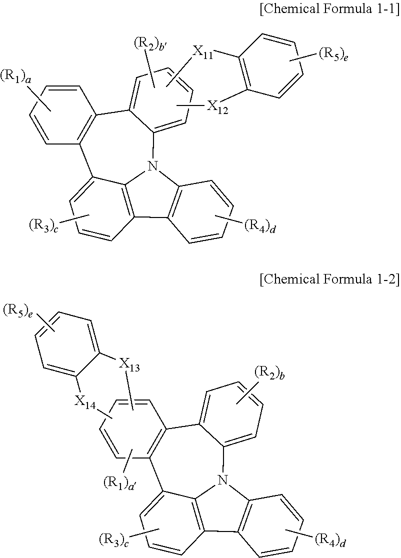
wherein in Chemical Formula 1-1 and Chemical Formula 1-2:
a to e have the same definitions as in Chemical Formulae 1 and 2;
any one of X₁₁ and X₁₂ is a direct bond, and the other one is CRaRb, O or S;
any one of X₁₃ and X₁₄ is a direct bond, and the other one is CRaRb, O or S;
Ra and Rb are the same as or different from each other, and each independently is hydrogen, deuterium, a substituted or unsubstituted alkyl group, a substituted or unsubstituted aryl group, a substituted or unsubstituted heteroaryl group, a substituted or unsubstituted cycloalkyl group, a substituted or unsubstituted alkoxy group or a combination thereof; and
at least one of R₁ to R₅ is the following Chemical Formula 3, and the rest that are not Chemical Formula 3 are the same as or different from each other and each independently is hydrogen, deuterium, a substituted or unsubstituted alkyl group, a substituted or unsubstituted aryl group, a substituted or unsubstituted heteroaryl group, a substituted or unsubstituted cycloalkyl group, a substituted or unsubstituted alkoxy group or a combination thereof; 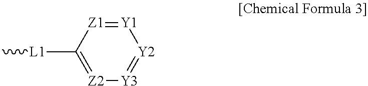
wherein in Chemical Formula 3: [symbol]
is a part bonding to the core of Chemical Formula 1-1 or 1-2;
at least one of Z1 and Z2 is N, and the other one is CRc;
Y1 to Y3 are the same as or different from each other, and each independently is CRd or N;
L1 is a direct bond, a substituted or unsubstituted alkylene group, a substituted or unsubstituted arylene group, a substituted or unsubstituted heteroarylene group or a combination thereof;
Rc and Rd have the same definitions as in claim 1;
a′ is an integer of 0 to 2, and when a′ is 2, the R₁s are the same as or different from each other; and
b′ is an integer of 0 to 2, and when b′ is 2, the R₂s are the same as or different from each other.

3. The heterocyclic compound of claim 1, wherein Chemical Formula 1 is the following Chemical Formula 1-3: 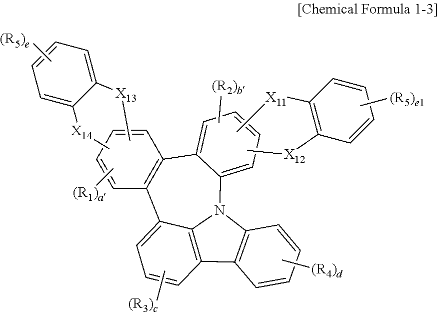
wherein in Chemical Formula 1-3:
c to e have the same definitions as in Chemical Formulae 1 and 2;
any one of X₁₁ and X₁₂ is a direct bond, and the other one is CRaRb, O or S;
any one of X₁₃ and X₁₄ is a direct bond, and the other one is CRaRb, O or S;
Ra and Rb are the same as or different from each other, and each independently is hydrogen, deuterium, a substituted or unsubstituted alkyl group, a substituted or unsubstituted aryl group, a substituted or unsubstituted heteroaryl group, a substituted or unsubstituted cycloalkyl group, a substituted or unsubstituted alkoxy group or a combination thereof, and
at least one of R₁ to R₅ and R₁₅ is the following Chemical Formula 3, and the rest that are not Chemical Formula 3 are the same as or different from each other and each independently is hydrogen, deuterium, a substituted or unsubstituted alkyl group, a substituted or unsubstituted aryl group, a substituted or unsubstituted heteroaryl group, a substituted or unsubstituted cycloalkyl group, a substituted or unsubstituted alkoxy group or a combination thereof: 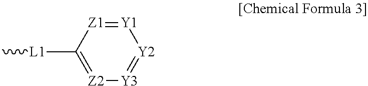
wherein in Chemical Formula 3: [symbol]
is a part bonding to the core of Chemical Formula 1-3;
at least one of Z1 and Z2 is N, and the other one is CRc;
Y1 to Y3 are the same as or different from each other, and each independently is CRd or N;
L1 is a direct bond, a substituted or unsubstituted alkylene group, a substituted or unsubstituted arylene group, a substituted or unsubstituted heteroarylene group or a combination thereof,
Rc and Rd have the same definitions as in claim 1;
e1 is an integer of 0 to 4, and when e1 is 2 or greater, the R₁₅s are the same as or different from each other;
a′ is an integer of 0 to 2, and when a′ is 2 or greater, the R₁s are the same as or different from each other; and
b′ is an integer of 0 to 2, and when b′ is 2 or greater, the R₂s are the same as or different from each other.

4. The heterocyclic compound of claim 1, wherein Chemical Formula 1 is any one of the following Chemical Formula 1-4 to Chemical Formula 1-11: 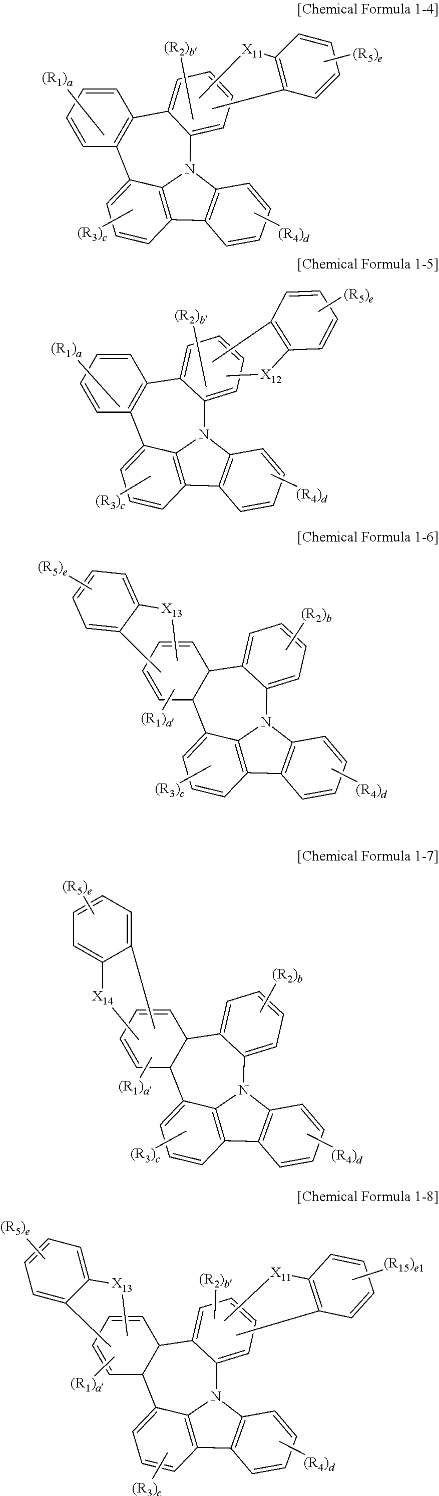 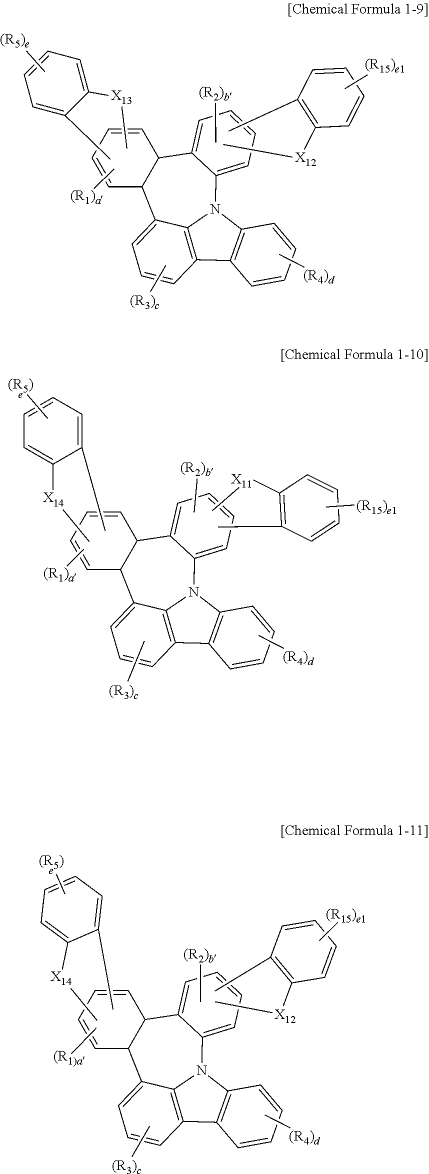
wherein in Chemical Formula 1-4 to Chemical Formula 1-11;
X₁₁ and X₁₂ are each independently CRaRb, O or S;
any one of X₁₃ and X₁₄ are each independently CRaRb, O or S;
Ra and Rb are the same as or different from each other, and each independently is hydrogen, deuterium, a substituted or unsubstituted alkyl group, a substituted or unsubstituted aryl group, a substituted or unsubstituted heteroaryl group, a substituted or unsubstituted cycloalkyl group, a substituted or unsubstituted alkoxy group or a combination thereof;
at least one of R₁ to R₅ and R₁₅ is the following Chemical Formula 3, and the rest that are not Chemical Formula 3 are the same as or different from each other and each independently is hydrogen, deuterium, a substituted or unsubstituted alkyl group, a substituted or unsubstituted aryl group, a substituted or unsubstituted heteroaryl group, a substituted or unsubstituted cycloalkyl group, a substituted or unsubstituted alkoxy group or a combination thereof,
a is an integer of 0 to 4, and when a is 2 or greater, the R₁s are the same as or different from each other;
b is an integer of 0 to 4, and when b is 2 or greater, the R₂s are the same as or different from each other;
c is an integer of 0 to 3, and when c is 2 or greater, the R₃s are the same as or different from each other;
d is an integer of 0 to 4, and when d is 2 or greater, the R₄s are the same as or different from each other;
e is an integer of 0 to 4, and when e is 2 or greater, the R₅s are the same as or different from each other;
e1 is an integer of 0 to 4, and when e1 is 2 or greater, the R₁₅s are the same as or different from each other;
a′ is an integer of 0 to 2, and when a′ is 2, the R₁s are the same as or different from each other; and
b′ is an integer of 0 to 2, and when b′ is 2, the R₂s are the same as or different from each other.

5. The heterocyclic compound of claim 1, wherein at least one of Y1 to Y3 is CRd, and Rd is hydrogen, an aryl group having 6 to 20 carbon atoms, or a heteroaryl group having 3 to 30 carbon atoms.

6. The heterocyclic compound of claim 1, wherein at least one pair of Rd adjacent to each other bond to form an aromatic ring or a heteroring.

7. The heterocyclic compound of claim 1, wherein Chemical Formula 1 is any one of the following compounds:

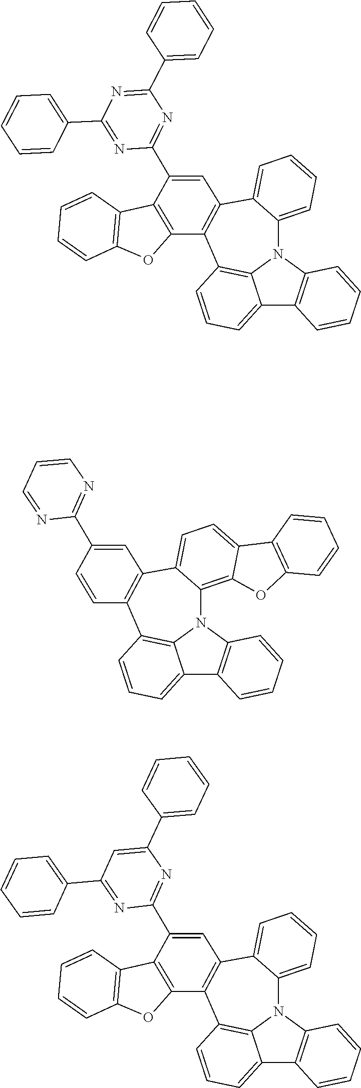 [SMILES: c1ccc(-c2nc(-c3ccccc3)nc(-c3cc4c(c5oc6ccccc6c35)-c3cccc5c6ccccc6n(c35)-c3ccccc3-4)n2)cc1 ; c1cnc(-c2ccc3c(c2)-c2ccc4c(oc5ccccc54)c2-n2c4ccccc4c4cccc-3c42)nc1 ; c1ccc(-c2cc(-c3ccccc3)nc(-c3cc4c(c5oc6ccccc6c35)-c3cccc5c6ccccc6n(c35)-c3ccccc3-4)n2)cc1]

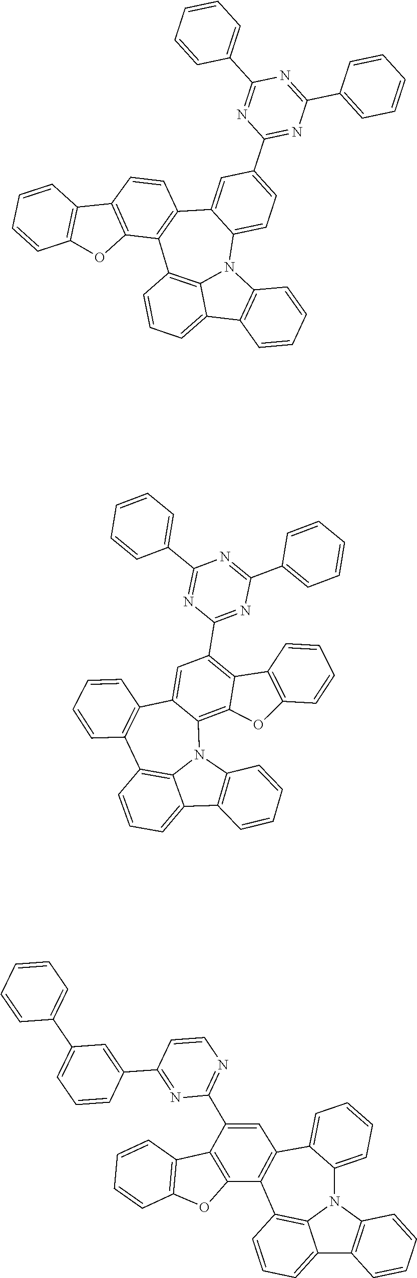 [SMILES: c1ccc(-c2nc(-c3ccccc3)nc(-c3ccc4c(c3)-c3ccc5c(oc6ccccc65)c3-c3cccc5c6ccccc6n-4c35)n2)cc1 ; c1ccc(-c2nc(-c3ccccc3)nc(-c3cc4c(c5oc6ccccc6c35)-n3c5ccccc5c5cccc(c53)-c3ccccc3-4)n2)cc1 ; c1ccc(-c2cccc(-c3ccnc(-c4cc5c(c6oc7ccccc7c46)-c4cccc6c7ccccc7n(c46)-c4ccccc4-5)n3)c2)cc1]

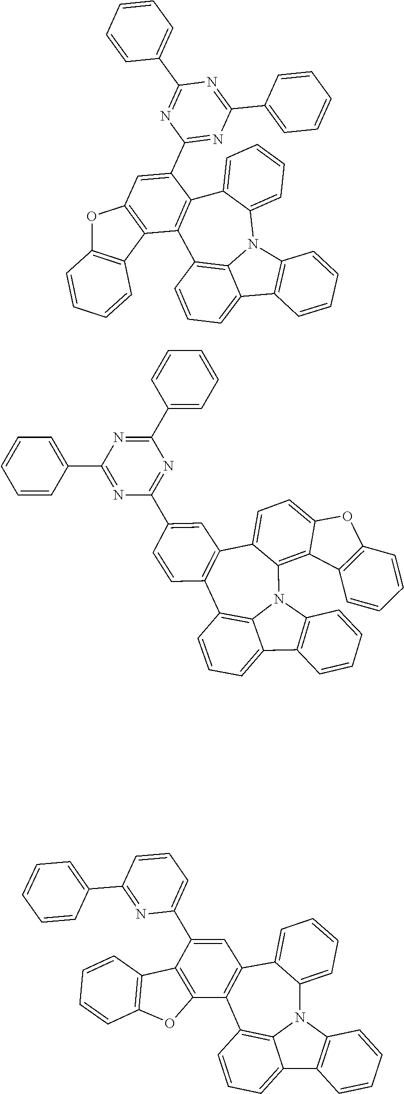

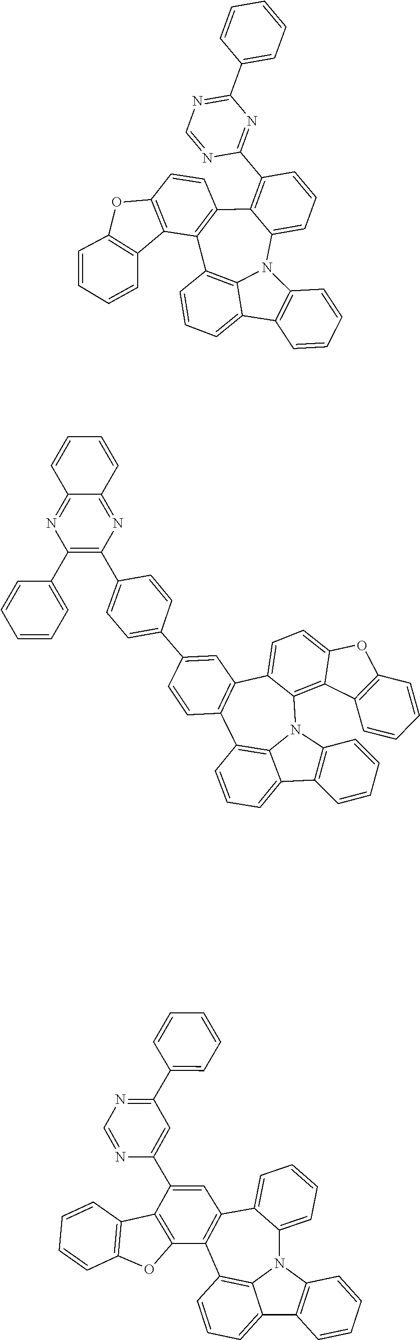 [SMILES: c1ccc(-c2ncnc(-c3cccc4c3-c3ccc5oc6ccccc6c5c3-c3cccc5c6ccccc6n-4c35)n2)cc1 ; c1ccc(-c2nc3ccccc3nc2-c2ccc(-c3ccc4c(c3)-c3ccc5oc6ccccc6c5c3-n3c5ccccc5c5cccc-4c53)cc2)cc1 ; c1ccc(-c2cc(-c3cc4c(c5oc6ccccc6c35)-c3cccc5c6ccccc6n(c35)-c3ccccc3-4)ncn2)cc1]

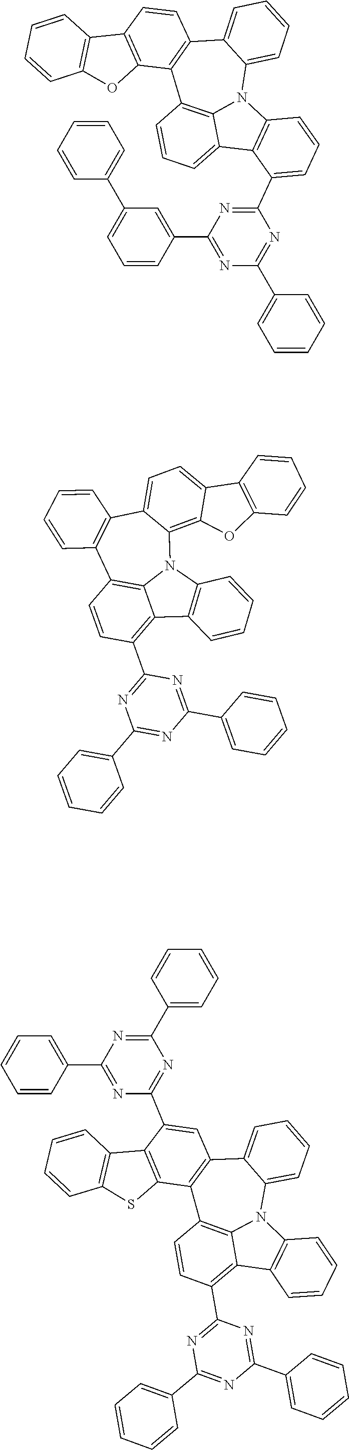 [SMILES: c1ccc(-c2cccc(-c3nc(-c4ccccc4)nc(-c4cccc5c4c4cccc6c4n5-c4ccccc4-c4ccc5c(oc7ccccc75)c4-6)n3)c2)cc1 ; c1ccc(-c2nc(-c3ccccc3)nc(-c3ccc4c5c3c3ccccc3n5-c3c(ccc5c3oc3ccccc35)-c3ccccc3-4)n2)cc1 ; c1ccc(-c2nc(-c3ccccc3)nc(-c3cc4c(c5sc6ccccc6c35)-c3ccc(-c5nc(-c6ccccc6)nc(-c6ccccc6)n5)c5c6ccccc6n(c35)-c3ccccc3-4)n2)cc1]

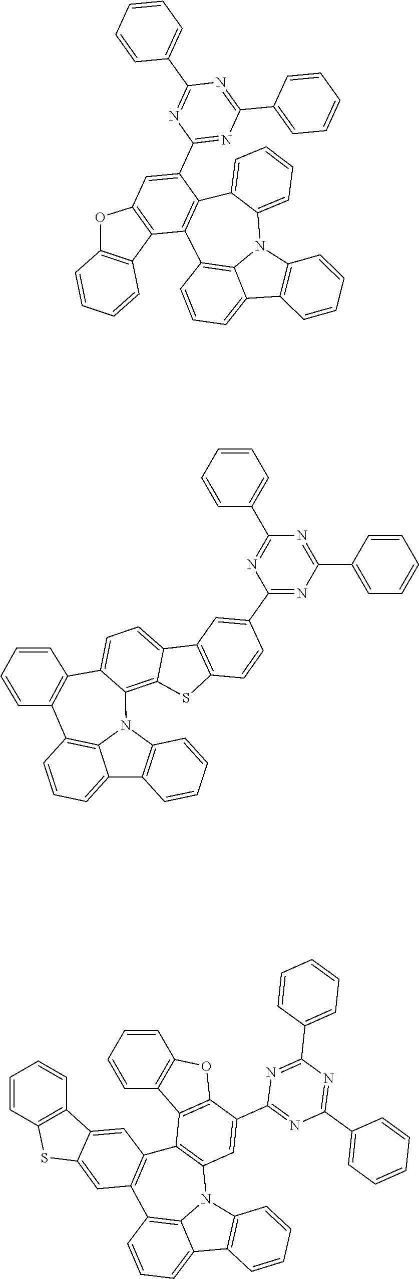 [SMILES: c1ccc(-c2nc(-c3ccccc3)nc(-c3cc4oc5ccccc5c4c4c3-c3ccccc3-n3c5ccccc5c5cccc-4c53)n2)cc1 ; c1ccc(-c2nc(-c3ccccc3)nc(-c3ccc4sc5c6c(ccc5c4c3)-c3ccccc3-c3cccc4c5ccccc5n-6c34)n2)cc1 ; c1ccc(-c2nc(-c3ccccc3)nc(-c3cc4c(c5c3oc3ccccc35)-c3cc5c(cc3-c3cccc6c7ccccc7n-4c36)sc3ccccc35)n2)cc1]

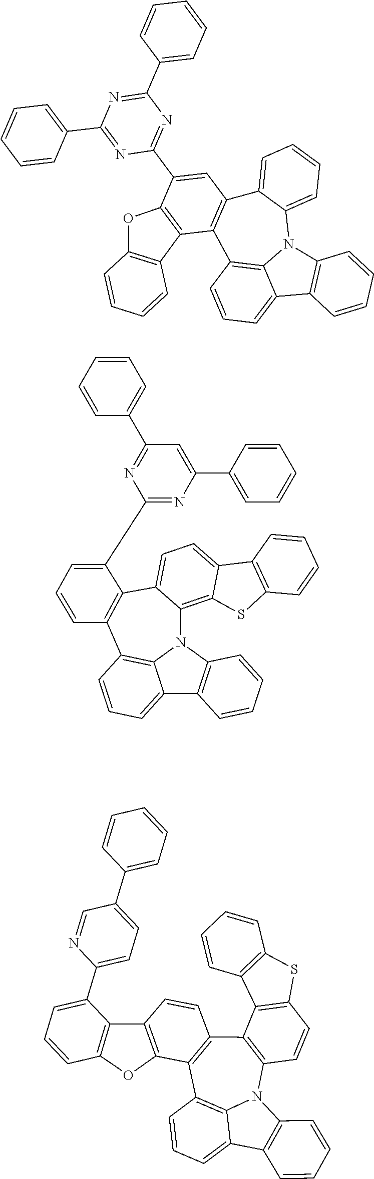

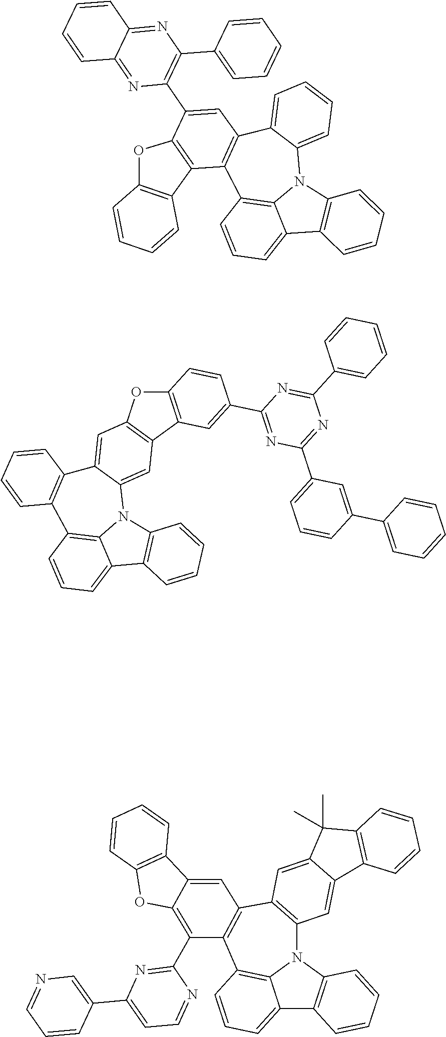 [SMILES: c1ccc(-c2nc3ccccc3nc2-c2cc3c(c4c2oc2ccccc24)-c2cccc4c5ccccc5n(c24)-c2ccccc2-3)cc1 ; c1ccc(-c2cccc(-c3nc(-c4ccccc4)nc(-c4ccc5oc6cc7c(cc6c5c4)-n4c5ccccc5c5cccc(c54)-c4ccccc4-7)n3)c2)cc1 ; CC1(C)c2ccccc2-c2cc3c(cc21)-c1cc2c(oc4ccccc42)c(-c2nccc(-c4cccnc4)n2)c1-c1cccc2c4ccccc4n-3c12]

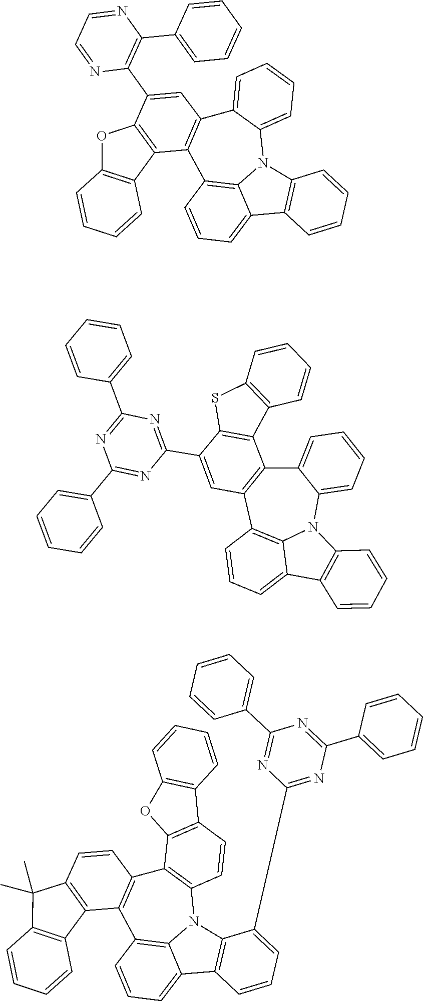 [SMILES: c1ccc(-c2nccnc2-c2cc3c(c4c2oc2ccccc24)-c2cccc4c5ccccc5n(c24)-c2ccccc2-3)cc1 ; c1ccc(-c2nc(-c3ccccc3)nc(-c3cc4c(c5c3sc3ccccc35)-c3ccccc3-n3c5ccccc5c5cccc-4c53)n2)cc1 ; CC1(C)c2ccccc2-c2c1ccc1c2-c2cccc3c4cccc(-c5nc(-c6ccccc6)nc(-c6ccccc6)n5)c4n(c23)-c2ccc3c(oc4ccccc43)c2-1]

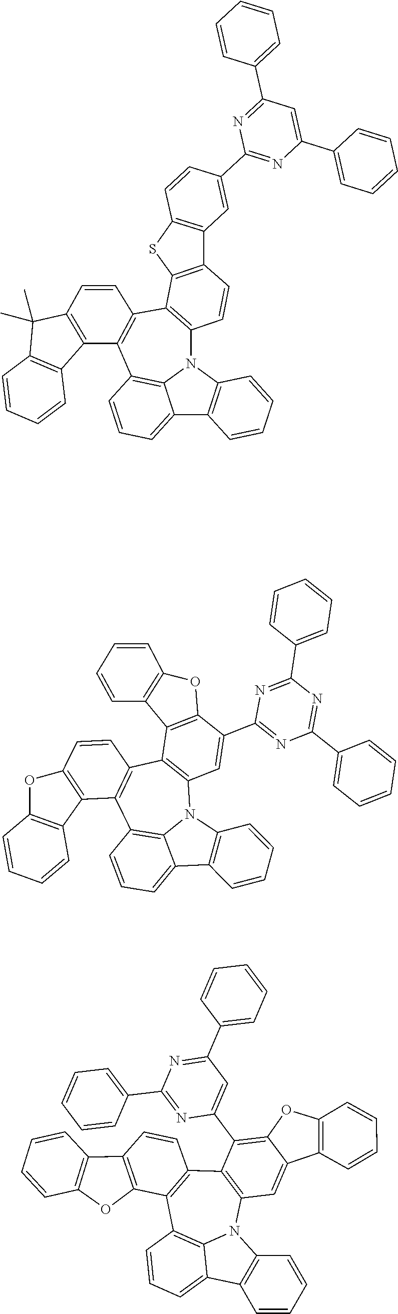 [SMILES: CC1(C)c2ccccc2-c2c1ccc1c2-c2cccc3c4ccccc4n(c23)-c2ccc3c(sc4ccc(-c5nc(-c6ccccc6)cc(-c6ccccc6)n5)cc43)c2-1 ; c1ccc(-c2nc(-c3ccccc3)nc(-c3cc4c(c5c3oc3ccccc35)-c3ccc5oc6ccccc6c5c3-c3cccc5c6ccccc6n-4c35)n2)cc1 ; c1ccc(-c2cc(-c3c4c(cc5c3oc3ccccc35)-n3c5ccccc5c5cccc(c53)-c3c-4ccc4c3oc3ccccc34)nc(-c3ccccc3)n2)cc1]

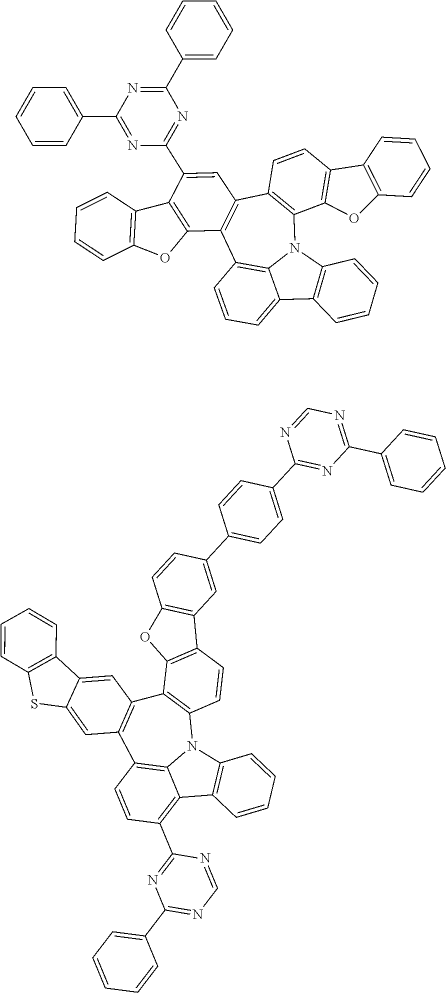 [SMILES: c1ccc(-c2nc(-c3ccccc3)nc(-c3cc4c(c5oc6ccccc6c35)-c3cccc5c6ccccc6n(c35)-c3c-4ccc4c3oc3ccccc34)n2)cc1 ; c1ccc(-c2ncnc(-c3ccc(-c4ccc5oc6c7c(ccc6c5c4)-n4c5ccccc5c5c(-c6ncnc(-c8ccccc8)n6)ccc(c54)-c4cc5sc6ccccc6c5cc4-7)cc3)n2)cc1]

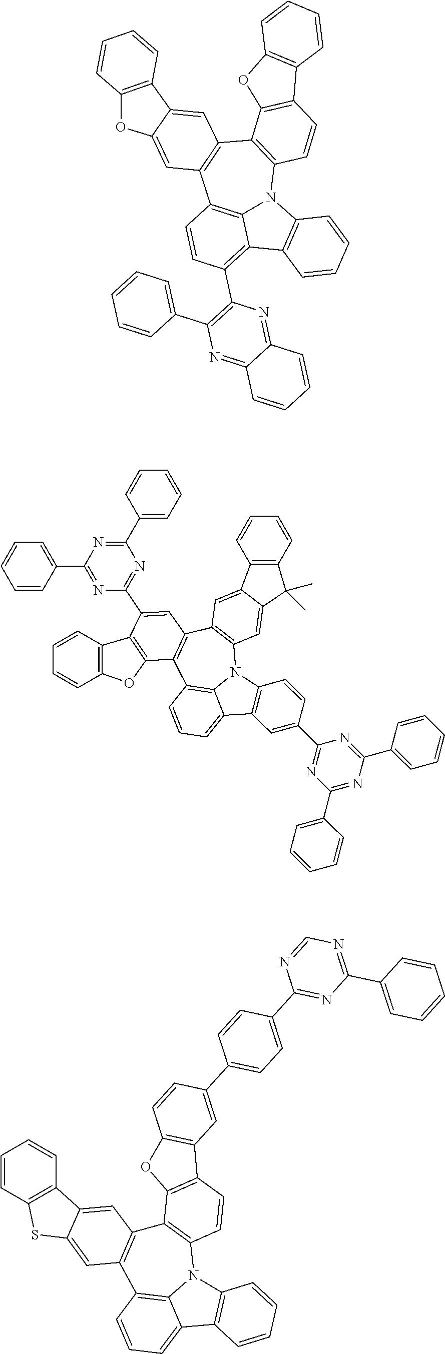 [SMILES: c1ccc(-c2nc3ccccc3nc2-c2ccc3c4c2c2ccccc2n4-c2ccc4c(oc5ccccc54)c2-c2cc4c(cc2-3)oc2ccccc24)cc1 ; CC1(C)c2ccccc2-c2cc3c(cc21)-n1c2ccc(-c4nc(-c5ccccc5)nc(-c5ccccc5)n4)cc2c2cccc(c21)-c1c-3cc(-c2nc(-c3ccccc3)nc(-c3ccccc3)n2)c2c1oc1ccccc12 ; c1ccc(-c2ncnc(-c3ccc(-c4ccc5oc6c7c(ccc6c5c4)-n4c5ccccc5c5cccc(c54)-c4cc5sc6ccccc6c5cc4-7)cc3)n2)cc1]

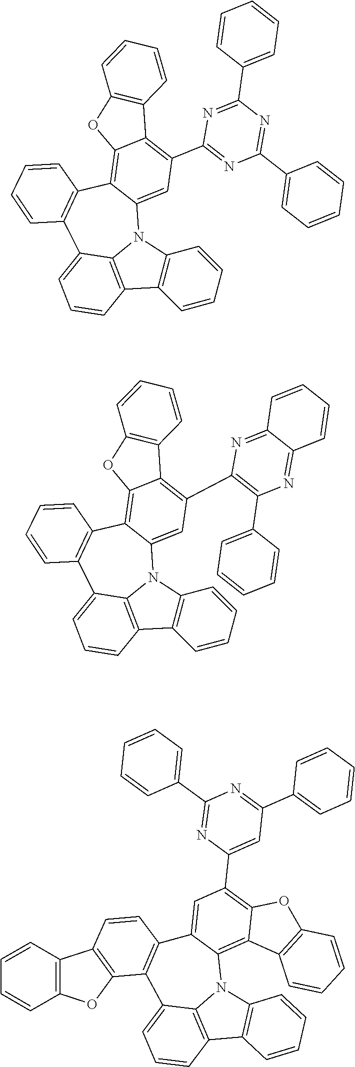 [SMILES: c1ccc(-c2nc(-c3ccccc3)nc(-c3cc4c(c5oc6ccccc6c35)-c3ccccc3-c3cccc5c6ccccc6n-4c35)n2)cc1 ; c1ccc(-c2nc3ccccc3nc2-c2cc3c(c4oc5ccccc5c24)-c2ccccc2-c2cccc4c5ccccc5n-3c24)cc1 ; c1ccc(-c2cc(-c3cc4c(c5c3oc3ccccc35)-n3c5ccccc5c5cccc(c53)-c3c-4ccc4c3oc3ccccc34)nc(-c3ccccc3)n2)cc1]

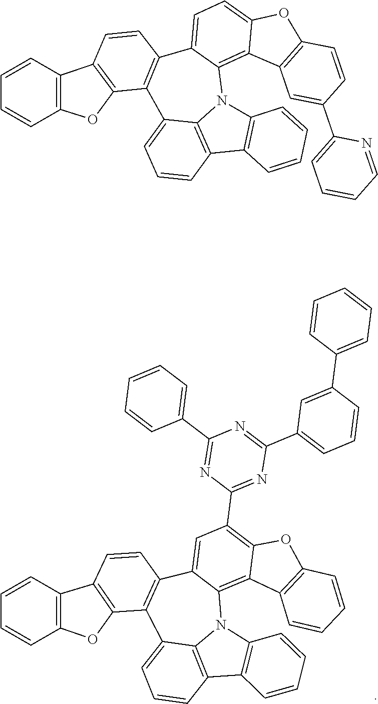 [SMILES: c1ccc(-c2ccc3oc4ccc5c(c4c3c2)-n2c3ccccc3c3cccc(c32)-c2c-5ccc3c2oc2ccccc23)nc1 ; c1ccc(-c2cccc(-c3nc(-c4ccccc4)nc(-c4cc5c(c6c4oc4ccccc46)-n4c6ccccc6c6cccc(c64)-c4c-5ccc5c4oc4ccccc45)n3)c2)cc1]

8. An organic light emitting device, comprising:
a first electrode;
a second electrode opposite to the first electrode; and
one or more organic material layers between the first electrode and the second electrode,
wherein one or more layers of the organic material layers include the heterocyclic compound of claim 1.

9. The organic light emitting device of claim 8, wherein the organic material layer includes a light emitting layer, and the light emitting layer includes the heterocyclic compound.

10. The organic light emitting device of claim 8, wherein the organic material layer includes a light emitting layer, and the light emitting layer includes the heterocyclic compound as a host of the light emitting layer.
